(19) United States
(12) Patent Application Publication
(10) Pub. No.: US 2025/0276953 A1
(43) Pub. Date: Sep. 4, 2025

(54) SMALL MOLECULE SIRTUIN INHIBITORS AND USES THEREOF

(71) Applicant: The Regents of the University of Michigan, Ann Arbor, MI (US)

(72) Inventors: Nouri Neamati, Ann Arbor, MI (US); Yanghan Liu, Ann Arbor, MI (US); Surinder Kumar, Ann Arbor, MI (US); David B. Lombard, Ann Arbor, MI (US)

(21) Appl. No.: 18/858,902

(22) PCT Filed: Apr. 28, 2023
(86) PCT No.: PCT/US2023/020366

§ 371 Date: Oct. 22, 2024

Related U.S. Application Data
(60) Provisional application No. 63/336,666, filed on Apr. 29, 2022.

(51) Int. Cl. C07D 277/42; A61K 47/55; C07D 277/48; C07F 9/6539

(52) U.S. Cl. CPC C07D 277/42; A61K 47/55; C07D 277/48; C07F 9/6539

(57) ABSTRACT

This invention is in the field of medicinal chemistry. In particular, the invention relates to a new class of small-molecules having a 2-hydroxybenzoic acid structure which function as sirtuin (e.g., SIRT1, SIRT2, SIRT3, SIRT4, SIRT5, SIRT6, SIRT7) inhibitors and/or degraders which function as effective therapeutic agents for treating, ameliorating, and preventing disorders associated with sirtuin activity (e.g., melanoma, Ewing sarcoma, malignant peripheral nerve sheath tumor, non-small cell lung cancer). In addition, this invention also relates to a new class of proteolysis-targeting chimeras (PROTACs) (as defined herein) which function as sirtuin inhibitors and/or degraders within cancer and/or immune cells. Pharmaceutical compositions comprising said compounds are also within the scope of the present invention.

### CROSS-REFERENCE TO RELATED APPLICATION

The present invention claims the priority benefit of U.S. Provisional Patent Application 63/336,666, filed Apr. 29, 2022, which is hereby incorporated by reference.

### STATEMENT REGARDING FEDERALLY SPONSORED RESEARCH OR DEVELOPMENT

This invention was made with government support under W81XWH-18-1-0567 awarded by the U.S. Army Medical Research and Development Command. The government has certain rights in the invention.

### FIELD OF THE INVENTION

This invention is in the field of medicinal chemistry. In particular, the invention relates to a new class of small-molecules having a 2-hydroxybenzoic acid structure which function as sirtuin (e.g., SIRT1, SIRT2, SIRT3, SIRT4, SIRT5, SIRT6, SIRT7) inhibitors and/or degraders which function as effective therapeutic agents for treating, ameliorating, and preventing disorders associated with sirtuin activity (e.g., melanoma, Ewing sarcoma, malignant peripheral nerve sheath tumor, non-small cell lung cancer). In addition, this invention also relates to a new class of proteolysis-targeting chimeras (PROTACs) (as defined herein) which function as sirtuin inhibitors and/or degraders within cancer and/or immune cells. Pharmaceutical compositions comprising said compounds are also within the scope of the present invention.

### INTRODUCTION

Sirtuins are known as nicotinamide adenine dinucleotide (NAD⁺)-dependent protein deacylases which are involved in many important biological functions, including the regulation of life span, transcription, DNA repair, protein secretion, and metabolism. There are seven sirtuin members encoded in mammalian genomes, SIRT1 through SIRT7, which are distinguished from another with respect to catalytic activities, subcellular localization, protein targets, and biological functions.

Sirtuin-family proteins are NAD⁺-dependent protein deacylases regulating metabolism and other diverse aspects of cell biology (Giblin et al., 2014). Three sirtuins (SIRT3, SIRT4, and SIRT5) reside primarily or exclusively in the mitochondrial matrix (Lombard et al., 2012). SIRT5 is an inefficient deacetylase, instead removing succinyl, malonyl, and glutaryl groups from lysines of its target proteins (referred to herein as Ksucc, Kmal, and Kglu, respectively) (Du et al., 2011; Park et al., 2013; Peng et al., 2011; Rardin et al., 2013; Tan et al., 2014). SIRT5 remains a poorly understood sirtuin, in part because most phenotypes of SIRT5 deficiency in normal cells and tissues are fairly modest. SIRT5 knockout (KO) mice are grossly unremarkable, fertile, and healthy until at least 18 months of age (Lombard et al., 2007). Germline SIRT5 KO mice show mild biochemical defects in response to a prolonged 48-hour fast (Nakagawa et al., 2009; Nishida et al., 2015; Rardin et al., 2013; Yu et al., 2013), and mild cardiac defects with advancing age (Sadhukhan et al., 2016). In contrast, SIRT5 KO mice actually show modest protection from the negative consequences of high fat diet (Yu et al., 2013).

Like other sirtuins, SIRT5 has been implicated in neoplasia, as both an oncogene and a tumor suppressor, in a context-specific manner (Kumar et al., 2018). As an oncogene, SIRT5 promotes folate metabolism via activation of mitochondrial serine hydroxymethyltransferase (SHMT2), facilitating cancer cell growth in vitro and in vivo (Yang et al., 2018). Folate metabolism is a target of several approved chemotherapy drugs. Likewise, SIRT5 inhibits pyruvate kinase muscle isozyme 2 (PKM2), resulting in accumulation of glycolytic intermediates, driving xenograft growth (Xiangyun et al., 2017). In colorectal cancer, SIRT5 promotes entry of glutamine into the TCA cycle by activating glutamate dehydrogenase 1 (GLUD1) (Wang et al., 2018). Additionally, SIRT5 desuccinylates citrate synthase (CS), the rate-limiting enzyme in the TCA cycle, promoting its activity (Ren et al., 2020). CS hyl ersucinylation inhibits its function and suppresses colorectal cancer cell proliferation and migration (Ren et al., 2020). In breast cancer, SIRT5 regulates glutamine metabolism by desuccinlVating glutaminase (GLS), protecting it from ubiquitin-mediated degradation (Greene et al., 2019). In melanoma, SIRT5 is required to maintain histone acetylation and methylation to promote expression of key genes, including MITF, a lineage-specific oncogene, and c-MYC (Giblin et al., 2021). Likewise, recent studies have documented oncogenic roles for SIRT5 in breast cancer and in AML (Yan et al., 2021; Abril et al., 2021).

Conversely, as a tumor suppressor, SIRT5 maintains fatty acid oxidation and redox homeostasis by inhibiting dimerization of acyl-CoA oxidase 1 (ACOX1), attenuating its function (Chen et al., 2018). Consistently, in hepatocellular carcinoma, low SIRT5 expression is associated with increased ACOX1 succinylation and activity (Chen et al., 2018). In AML, GBM, and certain other cancer types, isocitrate dehydrogenase (IDH) gain-of-function mutants convert α-ketoglutarate (α-KG) into the oncometabolite R-2-hydroxyglutarate (R-2HG), which in turn inhibits α-KG-dependent enzymes, including DNA and histone demethylases, thereby inducing epigenetic dysregulation (Kumar et al., 2018). Ectopic expression of SIRT5 reverses R-2HG-induced resistance to apoptosis in IDH1 mutant glioma cells, impairing their growth (Li et al., 2015). Recently, Hu and colleagues characterized a novel tumor suppressor function of SIRT5 in PDAC (Hu et al., 2021).

Proteolysis targeting chimeras (PROTACs) are a class of heterobifunctional molecules with two recruiting ligands connected via a linker. One ligand can recruit an E3 ubiquitin ligase while the other ligand specifically recruits the protein of interest (PoI). Upon ternary complex formation, PROTACs can recruit the E3 ligase to PoI in a spatially favorable presentation for substrate poly-ubiquitination, leading to subsequent degradation of PoI by the proteasome. In contrast to occupancy-based pharmacology, PROTACs technology offers several advantages. For example, it needs lower concentration than those occupancy-based inhibitors to achieve equivalent therapeutic effects. Besides, PROTACs can be used as a novel approach to target so called “undruggable” proteins, such as transcription factors, scaffolding proteins, and non-enzymatic proteins (Toure and Crews, 2016).

Therefore, SIRT5 is a new target for cancer therapy. SIRT5 inhibitors or degrades can be used as a single agent and in combination to treat various cancers as well as other diseases related to SIRT5 dysregulation.

New compounds capable of inhibiting SIRT5 activity or degrading SIRT5 are needed.

The present invention address this need.

### SUMMARY

Experiments conducted during the course of developing embodiments for the present invention involved design and discovery of novel 2-hydroxybenzoic acid derivatives as inhibitors and degraders of sirtuins. These compounds may be useful in the treatment of disorders associated with sirtuins, for example, cancer (melanoma, Ewing sarcoma, malignant peripheral nerve sheath tumor, non-small cell lung cancer).

Accordingly, the present invention provides a new class of small-molecules having a 2-hydroxybenzoic acid structure which function as sirtuin (e.g., SIRT1, SIRT2, SIRT3, SIRT4, SIRT5, SIRT6, SIRT7) inhibitors and/or degraders which function as effective therapeutic agents for treating, ameliorating, and preventing disorders associated with sirtuin activity (e.g., melanoma, Ewing sarcoma, malignant peripheral nerve sheath tumor, non-small cell lung cancer). In addition, this invention also relates to a new class of proteolysis-targeting chimeras (PROTACs) (as defined herein) which function as sirtuin inhibitors and/or degraders within cancer and/or immune cells. Pharmaceutical compositions comprising said compounds are also within the scope of the present invention.

In a particular embodiment, compounds encompassed within Formula (1), Formula (2), or Formula (3) are provided:

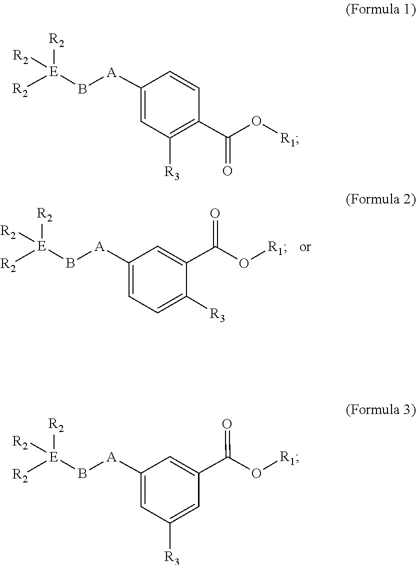

including pharmaceutically acceptable salts, solvates, PROTACs, and/or prodrugs thereof.

Formulas (1), (2), and (3) are not limited to a particular chemical moiety for R1, R2, R3, A, B and E. In some embodiments, the particular chemical moiety for R1, R2, R3, A, B and E independently include any chemical moiety that permits the resulting compound to inhibit and/or degrade sirtuin activity (e.g., SIRT1, SIRT2, SIRT3, SIRT4, SIRT5, SIRT6, SIRT7). In some embodiments, the particular chemical moiety for R1, R2, R3, A, B and E independently include any chemical moiety that permits the resulting compound to inhibit and/or degrade one or more of SIRT1, SIRT2, SIRT3, SIRT4, SIRT5, SIRT6, SIRT7. In some embodiments, the particular chemical moiety for R1, R2, R3, A, B and E independently include any chemical moiety that permits the resulting compound to inhibit and/or degrade SIRT5 activity. In some embodiments, the particular chemical moiety for R1, R2, R3, A, B and E independently include any chemical moiety that permits the resulting compound to inhibit and/or degrade sirtuin (e.g., SIRT5) related desuccinylase activity. In some embodiments, the particular chemical moiety for R1, R2, R3, A, B and E independently include any chemical moiety that permits the resulting compound to inhibit and/or degrade sirtuin (e.g., SIRT5) related demalonylase activity. In some embodiments, the particular chemical moiety for R1, R2, R3, A, B and E independently include any chemical moiety that permits the resulting compound to inhibit and/or degrade sirtuin (e.g., SIRT5) related deglutarylase activity. In some embodiments, the particular chemical moiety for R1, R2, R3, A, B and E independently include any chemical moiety that permits the resulting compound to influence multiple cellular pathways related to sirtuin activity (e.g., SIRT5) such as ammonia detoxification, fatty acid oxidation, cellular respiration, ketone body formation, tricarboxylic acid cycle (TCA), glycolysis and reactive oxygen species (ROS) metabolism. In some embodiments, the particular chemical moiety for R1, R2, R3, A, B and E independently include any chemical moiety that permits the resulting compound to serve as a therapeutic for treating, preventing and/or ameliorating disorders characterized with sirtuin (e.g., SIRT5) activity (e.g., sirtuin related cancer (e.g., melanoma, Ewing sarcoma, malignant peripheral nerve sheath tumor, non-small cell lung cancer).

In some embodiments, R¹ is selected from the group consisting of hydrogen, alkyl, cycloalkyl, heteroalkyl, heterocycloalkyl, aryl, heteroaryl, arylalkyl, and heteroarylalkyl.

In some embodiments, R¹ is selected from the group consisting of hydrogen, methyl, ethyl, n-propyl, isopropyl, n-butyl, isobutyl, sec-butyl, tert-butyl, pentyl,

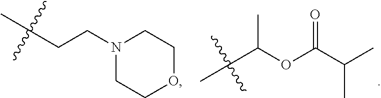

In some embodiments, A is selected from the group consisting of NH, CH₂, O, S,

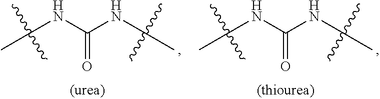 [SMILES: *NC(=O)N* ; *NC(=O)N*]

amide, and sulfonamide.

In some embodiments, B is an aromatic ring.

In some embodiments, B is a thiazole ring (e.g.

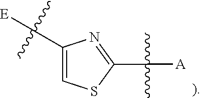

In some embodiments, E is an aromatic ring.

In some embodiments, E is a benzene ring.

In some embodiments,

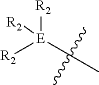

is selected from:

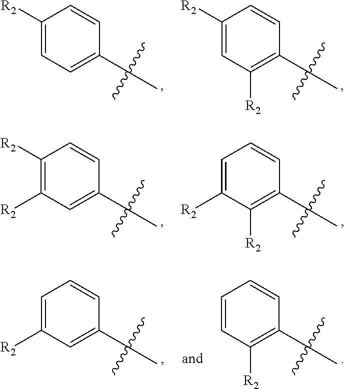

In some embodiments, each R² within

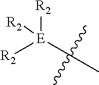

is independently selected from the group consisting of hydrogen, halogen, (e.g., F, Cl, Br, I), alkyl, cycloalkyl, heteroalkyl, heterocycloalkyl, halogen, aryl, heteroaryl, arylalkyl, heteroarylalkyl, nitryl, cyano, amide or sulfonamide,

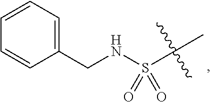 [SMILES: *S(=O)(=O)NCc1ccccc1]

and triphenylphosphine (TPP) attached with linker; and/or
wherein each R² within

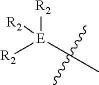

is independently selected from the group consisting of hydrogen, halogen, CF₃, OCH₃, OH, NO₂ (nitryl), CN (cyano), amide (e.g., NHC(O)CH₃), and sulfonamide (e.g., S(O2)NHCH3),

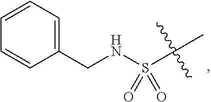 [SMILES: *S(=O)(=O)NCc1ccccc1]

and triphenylphosphine (TPP) group attached with a linker.

In some embodiments, linker is covalently bonded to connect two parts; the carbon atom in the linear chain can be substituted with oxygen, nitrogen, sulfur, ester, and amide.

In some embodiments, linker is of following Formula (L0):

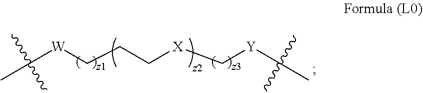

or an enantiomer, diastereomer, or stereoisomer thereof, wherein z1 is an integer selected from 0 to 10; Z2 is an integer selected from 0 to 10; Z3 is an integer selected from 0 to 10; each X is independently absent, CH₂, O, S, NH, NR₁₃; wherein W is selected from absent, O, NH, NR₁₃, —OCH₂C(O)NH—, —CH₂CH₂C(O)NH—, —CH₂C(O)NH— or —C(O)NH—; wherein Y is absent, O, NH, NR₁₃, —OCH₂C(O)NH—, —CH₂CH₂C(O)NH—, —CH₂C(O)NH— or —C(O)NH—; and each R₁₃ is independently C₁-C₃ alkyl. The linker can be substituted with an alkyl, halide, phenyl, benzyl, aryl, alkylene or heterocycle group.

In some embodiments, linker is one of the following moieties:

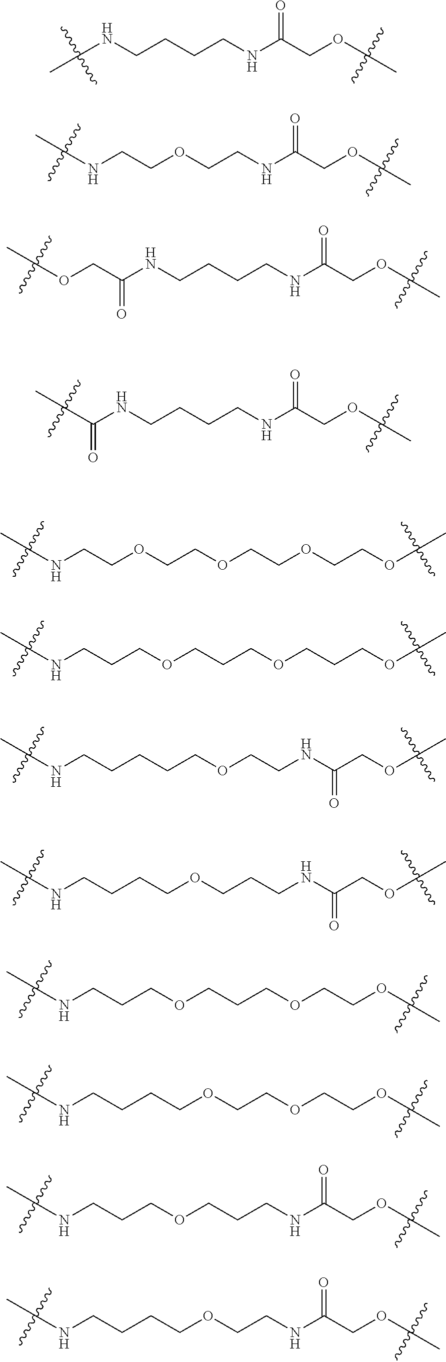

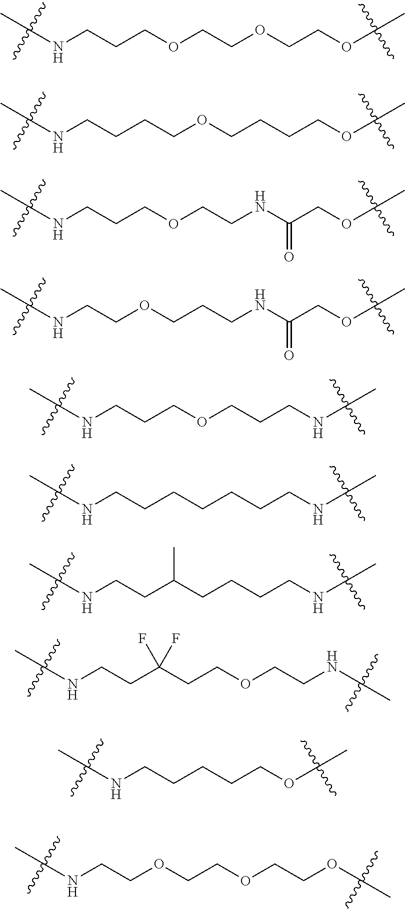

In some embodiments, R² is represented by Formula (P0):

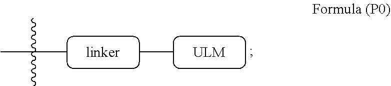

wherein the linker is covalently bonded to connect two parts; wherein the carbon atom in the linear chain can be substituted with oxygen, nitrogen, sulfur, ester, and amide; wherein ULM represents an E3 ubiquitin ligase binding moiety that binds E3 ubiquitin ligase selected from the group consisting of pomalidomide, thalidomide, lenalidomide, Von Hippel-Lindau (VHL), inhibitors of apoptosis proteins (LAP), Cereblon, and mouse double minute 2 (MDM2).

In some embodiments, R3 is hydrogen or OH.

In some embodiments, prodrug is preferably carboxylic acid ester. The ester group masks the negative charge on the carboxylate and thus can increase the cell permeability of the compounds. Inside the cells, the ester group can be hydrolyzed to release the negatively charged carboxylate, which can inhibit SIRT5.

In some embodiments, compounds shown in Table I are contemplated for Formula (1). Table I additionally shows values for SIRT5 IC₅₀ for select compounds.

TABLE I

| | | SIRT5 |
|---|---|---|
| Com- | | IC₅₀ |
| pound | Structure | (μM) |
| 1-1 | 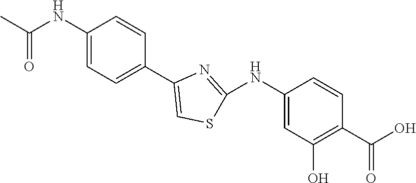 [SMILES: CC(=O)Nc1ccc(-c2csc(Nc3ccc(C(=O)O)c(O)c3)n2)cc1] | 26.45 ± 0.83 |
| 1-2 | 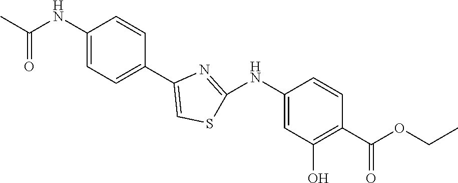 [SMILES: CCOC(=O)c1ccc(Nc2nc(-c3ccc(NC(C)=O)cc3)cs2)cc1O] | NA |
| 1-3 | 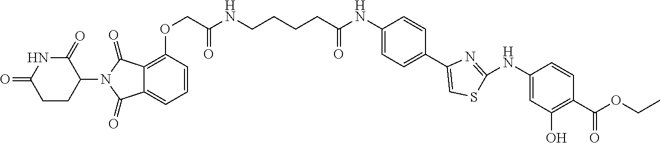 [SMILES: CCOC(=O)c1ccc(Nc2nc(-c3ccc(NC(=O)CCCCNC(=O)COc4cccc5c4C(=O)N(C4CCC(=O)NC4=O)C5=O)cc3)cs2)cc1O] | NA |
| 1-4 | 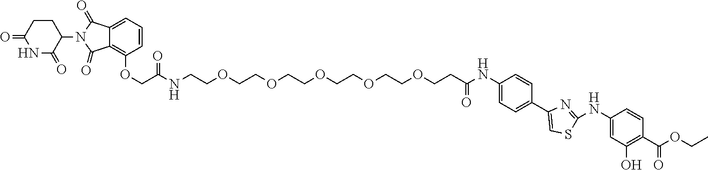 [SMILES: CCOC(=O)c1ccc(Nc2nc(-c3ccc(NC(=O)CCOCCOCCOCCOCCOCCNC(=O)COc4cccc5c4C(=O)N(C4CCC(=O)NC4=O)C5=O)cc3)cs2)cc1O] | NA |
| 1-5 | 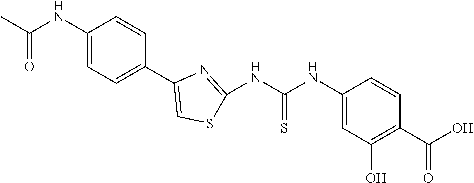 [SMILES: CC(=O)Nc1ccc(-c2csc(NC(=S)Nc3ccc(C(=O)O)c(O)c3)n2)cc1] | 12.43 ± 0.58 |
| 1-6 | 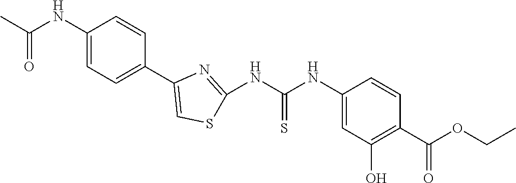 [SMILES: CCOC(=O)c1ccc(NC(=S)Nc2nc(-c3ccc(NC(C)=O)cc3)cs2)cc1O] | NA |
| 1-7 | 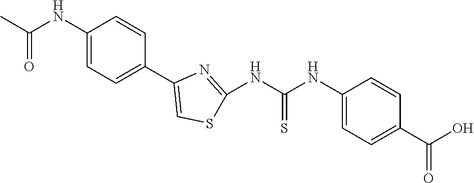 [SMILES: CC(=O)Nc1ccc(-c2csc(NC(=S)Nc3ccc(C(=O)O)cc3)n2)cc1] | 101.2 ± 8.48 |
| 1-8 | 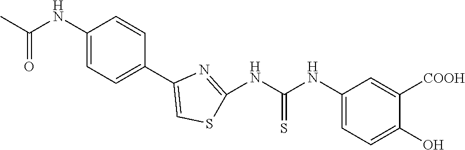 [SMILES: CC(=O)Nc1ccc(-c2csc(NC(=S)Nc3ccc(O)c(C(=O)O)c3)n2)cc1] | 55.94 ± 5.42 |
| 1-9 | 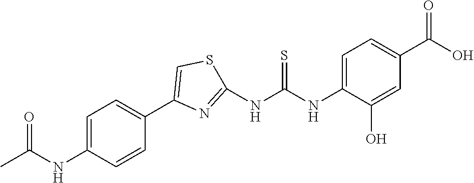 [SMILES: CC(=O)Nc1ccc(-c2csc(NC(=S)Nc3ccc(C(=O)O)cc3O)n2)cc1] | 50.72 ± 5.64 |
| 1-10 | 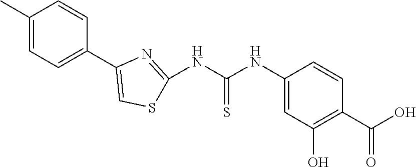 [SMILES: Cc1ccc(-c2csc(NC(=S)Nc3ccc(C(=O)O)c(O)c3)n2)cc1] | 23.45 ± 0.92 |
| 1-11 | 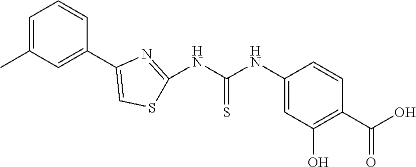 [SMILES: Cc1cccc(-c2csc(NC(=S)Nc3ccc(C(=O)O)c(O)c3)n2)c1] | 27.74 ± 1.02 |
| 1-12 | 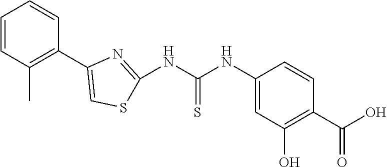 [SMILES: Cc1ccccc1-c1csc(NC(=S)Nc2ccc(C(=O)O)c(O)c2)n1] | 34.05 ± 2.18 |
| 1-13 | 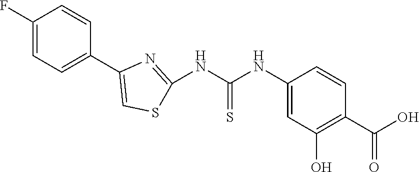 [SMILES: O=C(O)c1ccc(NC(=S)Nc2nc(-c3ccc(F)cc3)cs2)cc1O] | 17.26 ± 0.28 |
| 1-14 | 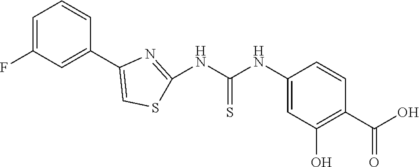 [SMILES: O=C(O)c1ccc(NC(=S)Nc2nc(-c3cccc(F)c3)cs2)cc1O] | 18.34 ± 0.95 |
| 1-15 | 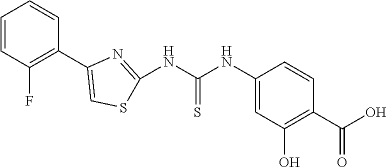 [SMILES: O=C(O)c1ccc(NC(=S)Nc2nc(-c3ccccc3F)cs2)cc1O] | 26.22 ± 2.54 |
| 1-16 | 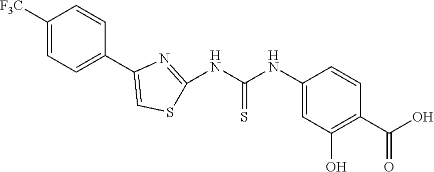 [SMILES: O=C(O)c1ccc(NC(=S)Nc2nc(-c3ccc(C(F)(F)F)cc3)cs2)cc1O] | 26.84 ± 1.42 |
| 1-17 | 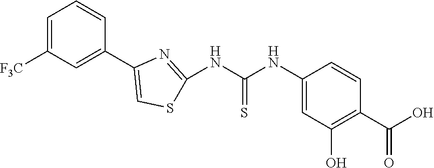 [SMILES: O=C(O)c1ccc(NC(=S)Nc2nc(-c3cccc(C(F)(F)F)c3)cs2)cc1O] | 24.27 ± 2.54 |
| 1-18 | 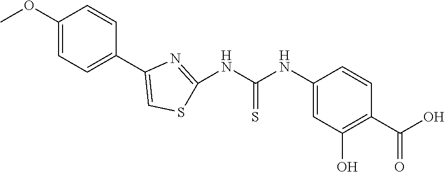 [SMILES: COc1ccc(-c2csc(NC(=S)Nc3ccc(C(=O)O)c(O)c3)n2)cc1] | 29.24 ± 2.88 |
| 1-19 | 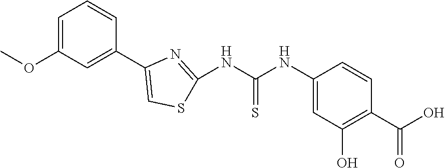 [SMILES: COc1cccc(-c2csc(NC(=S)Nc3ccc(C(=O)O)c(O)c3)n2)c1] | 41.62 ± 3.79 |
| 1-20 | 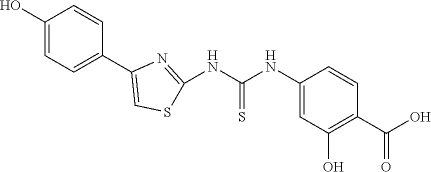 [SMILES: O=C(O)c1ccc(NC(=S)Nc2nc(-c3ccc(O)cc3)cs2)cc1O] | 42.54 ± 3.83 |
| 1-21 | 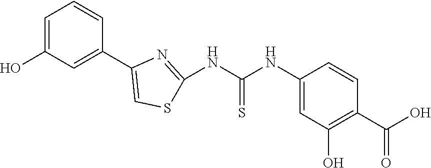 [SMILES: O=C(O)c1ccc(NC(=S)Nc2nc(-c3cccc(O)c3)cs2)cc1O] | 32.79 ± 3.87 |
| 1-22 | 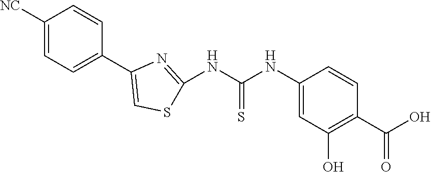 [SMILES: N#Cc1ccc(-c2csc(NC(=S)Nc3ccc(C(=O)O)c(O)c3)n2)cc1] | 11.42 ± 0.50 |
| 1-23 | 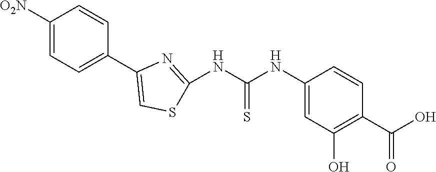 [SMILES: O=C(O)c1ccc(NC(=S)Nc2nc(-c3ccc([N+](=O)[O-])cc3)cs2)cc1O] | 4.3 ± 0.30 |
| 1-24 | 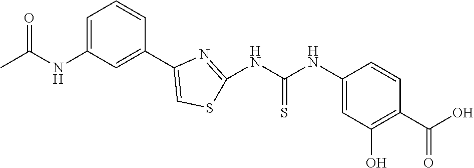 [SMILES: CC(=O)Nc1cccc(-c2csc(NC(=S)Nc3ccc(C(=O)O)c(O)c3)n2)c1] | 35.58 ± 1.30 |
| 1-25 | 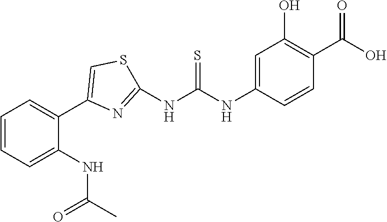 [SMILES: CC(=O)Nc1ccccc1-c1csc(NC(=S)Nc2ccc(C(=O)O)c(O)c2)n1] | 55.64 ± 5.49 |
| 1-26 | 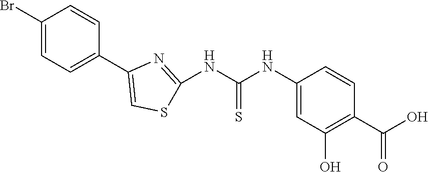 [SMILES: O=C(O)c1ccc(NC(=S)Nc2nc(-c3ccc(Br)cc3)cs2)cc1O] | 18.56 ± 0.23 |
| 1-27 | 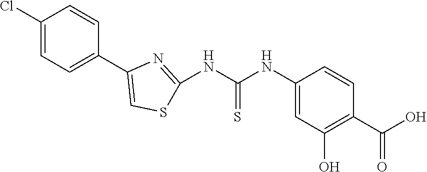 [SMILES: O=C(O)c1ccc(NC(=S)Nc2nc(-c3ccc(Cl)cc3)cs2)cc1O] | 18.41 ± 0.28 |
| 1-28 | 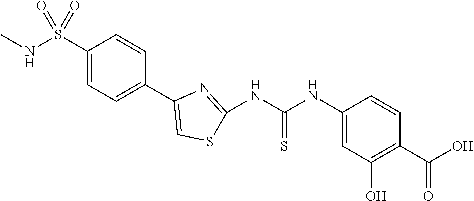 [SMILES: CNS(=O)(=O)c1ccc(-c2csc(NC(=S)Nc3ccc(C(=O)O)c(O)c3)n2)cc1] | 38.84 ± 3.13 |
| 1-29 | 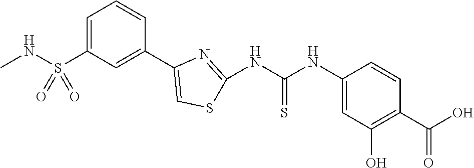 [SMILES: CNS(=O)(=O)c1cccc(-c2csc(NC(=S)Nc3ccc(C(=O)O)c(O)c3)n2)c1] | 26.14 ± 1.91 |
| 1-30 | 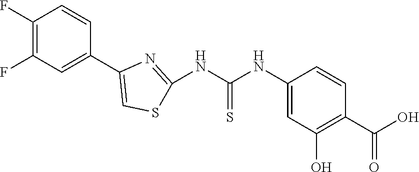 [SMILES: O=C(O)c1ccc(NC(=S)Nc2nc(-c3ccc(F)c(F)c3)cs2)cc1O] | 16.14 ± 0.73 |
| 1-31 | 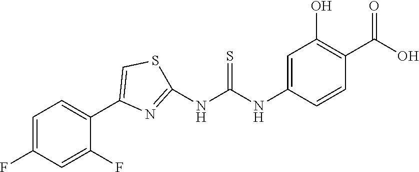 [SMILES: O=C(O)c1ccc(NC(=S)Nc2nc(-c3ccc(F)cc3F)cs2)cc1O] | 19.32 ± 0.93 |
| 1-32 | 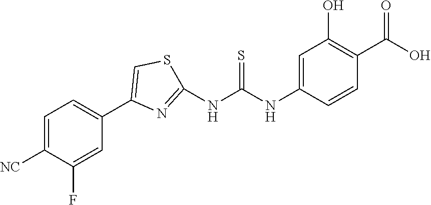 [SMILES: N#Cc1ccc(-c2csc(NC(=S)Nc3ccc(C(=O)O)c(O)c3)n2)cc1F] | 12.78 ± 0.48 |
| 1-33 | 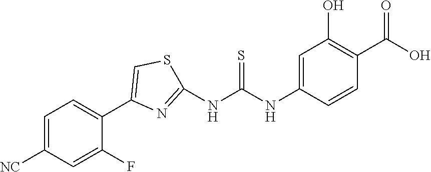 [SMILES: N#Cc1ccc(-c2csc(NC(=S)Nc3ccc(C(=O)O)c(O)c3)n2)c(F)c1] | 14.79 ± 0.81 |
| 1-34 | 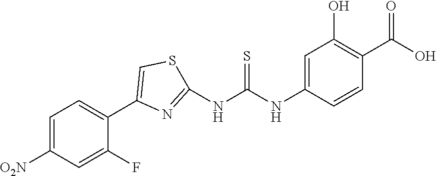 [SMILES: O=C(O)c1ccc(NC(=S)Nc2nc(-c3ccc([N+](=O)[O-])cc3F)cs2)cc1O] | 23.76 ± 2.71 |
| 1-35 | 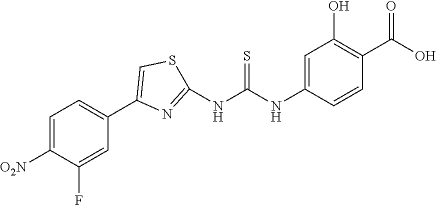 [SMILES: O=C(O)c1ccc(NC(=S)Nc2nc(-c3ccc([N+](=O)[O-])c(F)c3)cs2)cc1O] | 14.93 ± 1.35 |
| 1-36 | 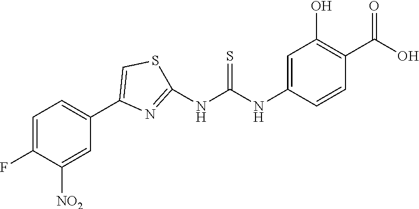 [SMILES: O=C(O)c1ccc(NC(=S)Nc2nc(-c3ccc(F)c([N+](=O)[O-])c3)cs2)cc1O] | 11.58 ± 0.74 |
| 1-37 | 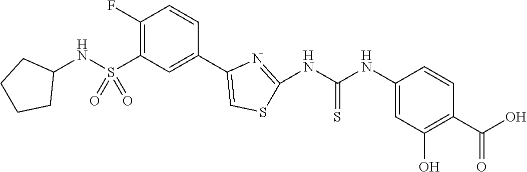 [SMILES: O=C(O)c1ccc(NC(=S)Nc2nc(-c3ccc(F)c(S(=O)(=O)NC4CCCC4)c3)cs2)cc1O] | 24.86 ± 0.54 |
| 1-38 | 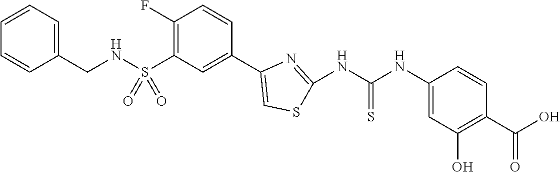 [SMILES: O=C(O)c1ccc(NC(=S)Nc2nc(-c3ccc(F)c(S(=O)(=O)NCc4ccccc4)c3)cs2)cc1O] | 8.22 ± 1.3 |
| 1-39 | 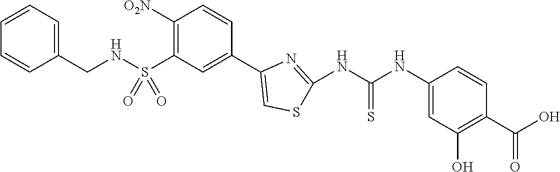 [SMILES: O=C(O)c1ccc(NC(=S)Nc2nc(-c3ccc([N+](=O)[O-])c(S(=O)(=O)NCc4ccccc4)c3)cs2)cc1O] | 2.5 ± 0.21 |
| 1-40 | 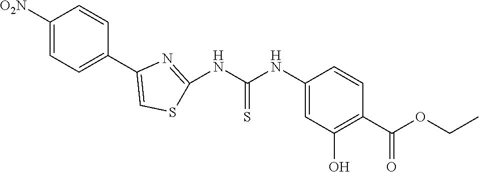 [SMILES: CCOC(=O)c1ccc(NC(=S)Nc2nc(-c3ccc([N+](=O)[O-])cc3)cs2)cc1O] | NA |
| 1-41 | 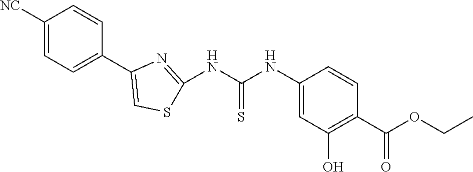 [SMILES: CCOC(=O)c1ccc(NC(=S)Nc2nc(-c3ccc(C#N)cc3)cs2)cc1O] | NA |
| 1-42 | 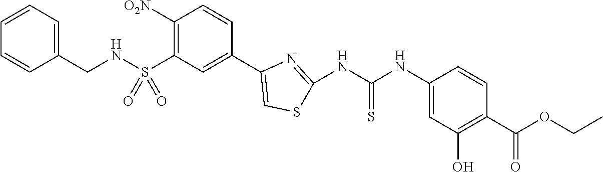 [SMILES: CCOC(=O)c1ccc(NC(=S)Nc2nc(-c3ccc([N+](=O)[O-])c(S(=O)(=O)NCc4ccccc4)c3)cs2)cc1O] | NA |
| 1-43 | 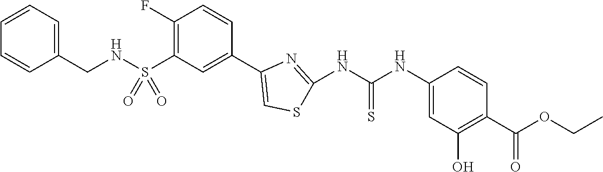 [SMILES: CCOC(=O)c1ccc(NC(=S)Nc2nc(-c3ccc(F)c(S(=O)(=O)NCc4ccccc4)c3)cs2)cc1O] | NA |
| 1-44 | 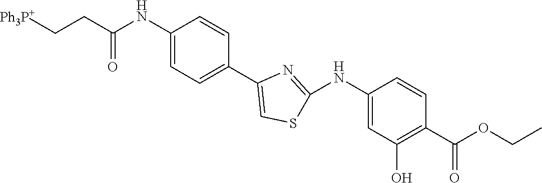 | NA |

The invention further provides processes for preparing any of the compounds of the present invention.

The invention also provides the use of compounds to not only inhibit sirtuin activity but also signaling pathways dependent upon or related to sirtuins. The invention also relates to the use of compounds for sensitizing cells to additional agent(s), such as agents known to be effective in the treatment of disorders related to sirtuin activity (e.g., cancer).

The compounds of the invention are useful for the treatment, amelioration, or prevention of disorders associated with sirtuin (e.g., SIRT5) activity (e.g., cancer), such as those responsive to sirtuin (e.g., SIRT5) activity inhibition (e.g., melanoma, Ewing sarcoma, malignant peripheral nerve sheath tumor, non-small cell lung cancer).

In certain embodiments, the present invention provides methods of treating, ameliorating, or preventing a disorder related to sirtuin (e.g., SIRT5) activity in a patient comprising administering to said patient a therapeutically effective amount of the pharmaceutical composition comprising a compound encompassed within Formulas 1, 2 or 3, and/or a compound recited in Table.

The invention also provides kits comprising a compound of the invention and instructions for administering the compound to an animal. The kits may optionally contain other therapeutic agents, e.g., agents useful in treating disorders related to sirtuin (e.g., SIRT5) activity (e.g., cancer).

### BRIEF DESCRIPTION OF THE DRAWINGS

FIG. 1A-B: (A) Structure of compound 1-2; (B) Compound 1-2 inhibited growth of Ewing sarcoma cell—A4573 and A673.

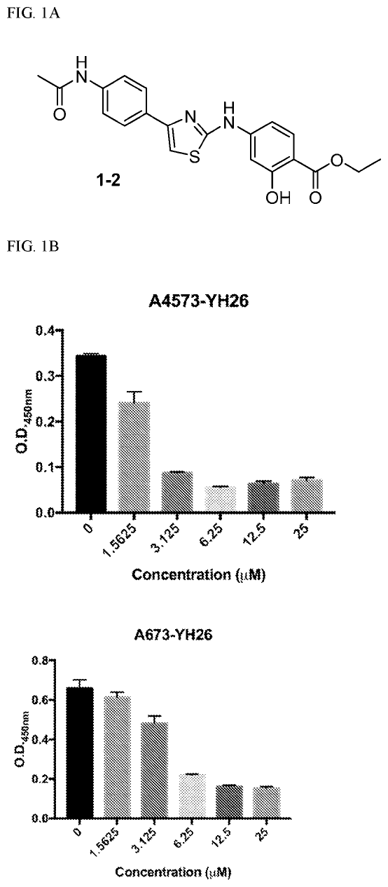

FIG. 2A-C: (A) Structures of compound 1-3; (B) Compound 1-3 degraded protein SIRT5; (C) Compound 1-3 inhibited growth of Ewing sarcoma cell lines A4573 and A673.

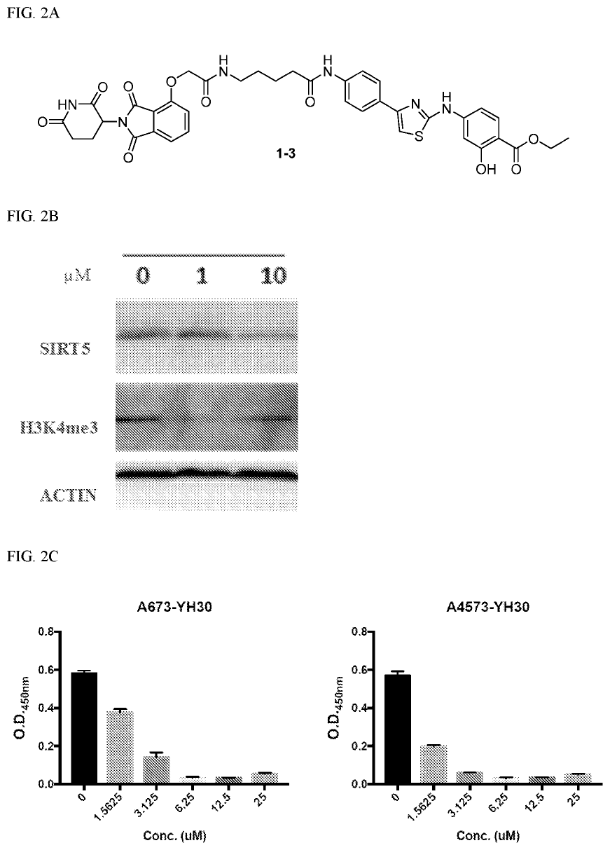

### DEFINITIONS

For convenience, before further description of the present invention, certain terms employed in the specification, examples, and appended claims are described here. These definitions should be read in light of the entire invention and as would be understood by a person skilled in the art.

As used herein, the term “alkyl” means a saturated hydrocarbon radical having 1 to 15 carbon atoms, preferably 1 to 10 carbon atoms, more preferably 1 to 5 carbon atoms, most preferably 1 to 3 carbon atoms, that may be branched or unbranched. Non-limiting examples of alkyl radicals include methyl, ethyl, n-propyl, isopropyl, n-butyl, isobutyl, sec-butyl, tert-butyl, pentyl, hexyl, heptyl, octyl and the like, wherein methyl, ethyl, n-propyl and isopropyl represent specifically preferred examples.

As used herein, the term “cycloalkyl” refers to a saturated hydrocarbon ring that is not aromatic. Cycloalkyl rings are monocyclic, or are fused, spiro, or bridged bicyclic or polycyclic ring systems. Monocyclic cycloalkyl rings contain from about 3 to about 12 carbon atoms, preferably from 3 to 7 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5-, 6- or 7-membered rings fused to 5-, 6- or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, cyclohexyl, cycloheptyl, cyclooctyl and cyclononyl rings.

As used herein, the term “halo” or “halogen” is fluoro, chloro, bromo or iodo. Preferred halo are fluoro, chloro and bromo.

As used herein, the term “heteroatom” is a nitrogen, sulfur, or oxygen atom. Groups containing more than one heteroatom may contain different heteroatoms.

As used herein, the term “heteroalkyl” is a saturated or unsaturated chain carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bounds and/or one or more carbon-carbon triple bounds. Preferred unsaturated heteroalkyl have one or two carbon-carbon double bounds or one carbon-carbon triple bound, more preferably one double bound. Heteroalkyl chains may be unsubstituted or substituted with from 1 to 4 substituents. Preferred substituted heteroalkyl are mono-, di, or tri-substituted. Heteroalkyl may be substituted with halo, cyano, alkyl, heteroalkyl, haloalkyl, phenyl, keto, hydroxy, carboxy, amino, acylamino, aryloxy, heteroaryloxy, or any combination thereof.

As used herein, the term “heterocycloalkyl” is a saturated or unsaturated ring containing carbon atoms and from 1 to 4 (preferably 1 to 3) heteroatoms in the ring. Heterocycloalkyl rings are monocyclic, or are fused, spiro, or bridged bicyclic or polycyclic ring systems. Monocyclic heterocycloalkyl rings contain from about 3 to about 9 member atoms (including both carbons and heteroatoms), preferably from 5 to 7 member atoms, in the ring. Bicyclic heterocycloalkyl rings may be fused, spiro, or bridged ring systems. Preferred bicyclic heterocycloalkyl rings comprise 5-, 6- or 7-membered rings fused to 5-, 6- or 7-membered rings. Heterocycloalkyl rings may be unsubstituted (i.e., contain hydrogen) or substituted (on either carbons or heteroatoms or both) with from 1 to 4 substituents selected from halo, cyano, alkyl, heteroalkyl, haloalkyl, phenyl, keto, hydroxy, carboxy, amino, acylamino, aryloxy, heteroaryloxy, or any combination thereof. Preferred heterocycloalkyl rings include, but are not limited to, any of the following:

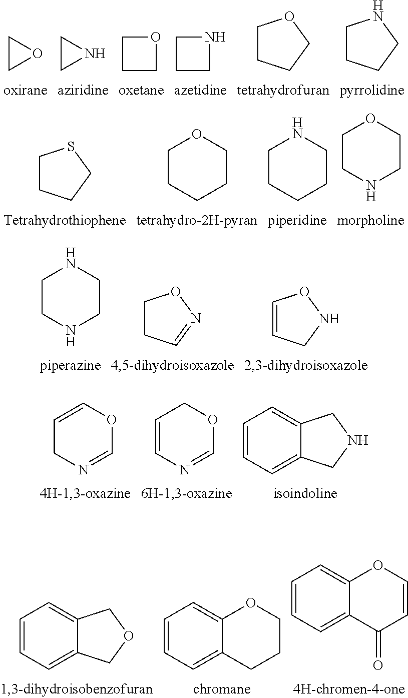

As used herein, the term “aromatic rings” include aromatic hydrocarbon rings and heteroaromatic rings. Aromatic rings may be unsubstituted or substituted with from 1 to 4 substituents. Preferred substituted aromatic rings are mono-, di, or tri-substituted. Aromatic rings may be substituted with halo, cyano, alkyl, heteroalkyl, haloalkyl, phenyl, keto, hydroxy, carboxy, amino, acylamino, aryloxy, heteroaryloxy, or any combination thereof. Preferred aromatic rings include, but are not limited to, the following:

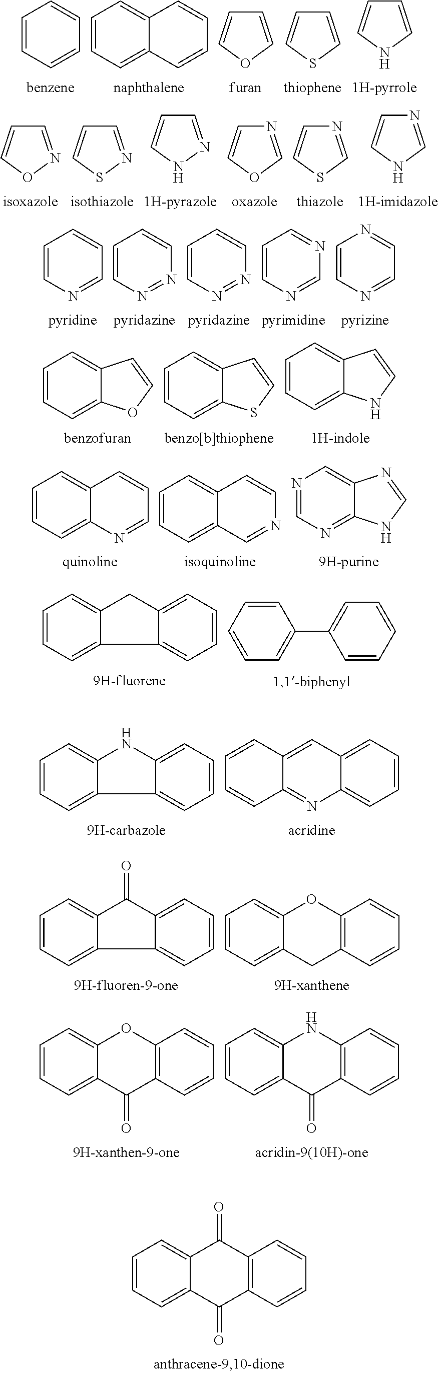

As used herein, the term “arylalkyl” or “aralkyl” alone or in combination, refers to an alkyl radical in which one hydrogen atom is replaced by an aryl radical, for example, benzyl and the like.

As used herein, the term “heteroarylalkyl” refers to an alkyl radical in which one hydrogen atom is replaced by a heteroaryl radical.

As used herein, the term “pharmacological composition” refers to a mixture of one or more of the compounds described herein or pharmaceutically acceptable salts thereof, with other chemical components, such as pharmaceutically acceptable carriers and/or excipients. The purpose of a pharmacological composition is to facilitate administration of a compound to an organism.

As used herein, the term “pharmaceutically acceptable salts” is a cationic salt formed at any acidic (e.g., carboxylic acid) group, or an anionic salt formed at any basic (e.g., amino) group.

As used herein, the term “linker” is covalently bonded to connect two parts; the carbon atom in the linear chain can be substituted with oxygen, nitrogen, sulfur, ester, and amide. Preferably, linker is of following Formula (L0):

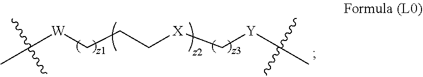

or an enantiomer, diastereomer, or stereoisomer thereof, wherein z1 is an integer selected from 0 to 10; Z2 is an integer selected from 0 to 10; Z3 is an integer selected from 0 to 10; each X is independently absent, CH₂, O, S, NH, NR₁₃: W is selected from absent, O, NH, NR₁₃, —OCH₂C(O)NH—, —CH₂CH₂C(O)NH—, —CH₂C(O)NH— or —C(O)NH—; wherein Y is absent, O, NH, NR₁₃, —OCH₂C(O)NH—, —CH₂CH₂C(O)NH—, —CH₂C(O)NH— or —C(O)NH—; and each R₁₃ is independently C₁-C₃ alkyl. The linker can be substituted with an alkyl, halide, phenyl, benzyl, aryl, alkylene or heterocycle group. Examples of applicable linkers include, but are not limited to, the following:

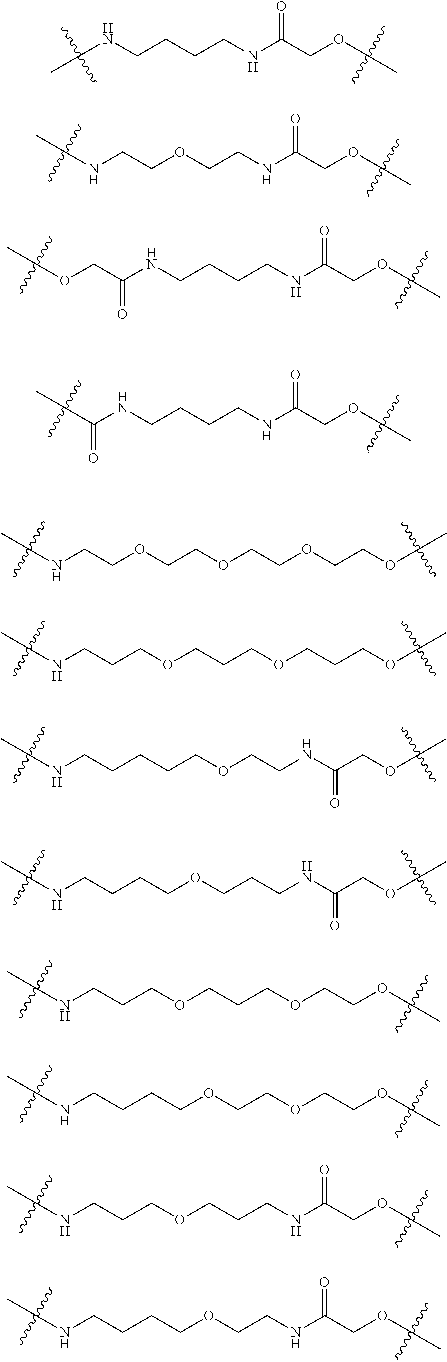

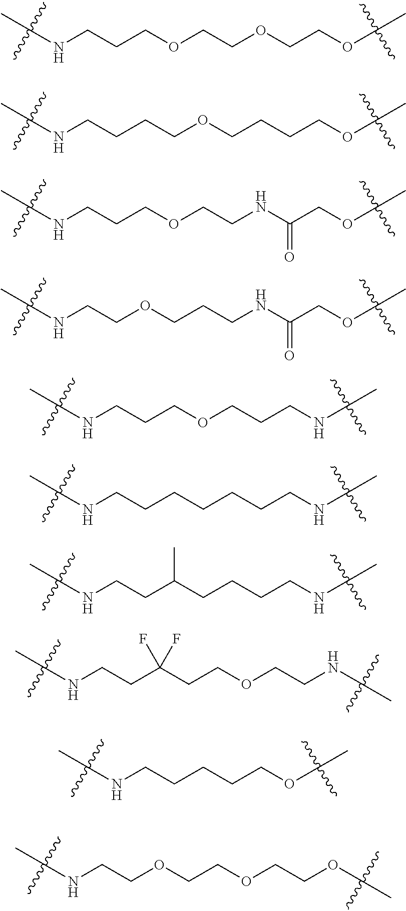

As used herein, the term “ULM” represents a E3 ubiquitin ligase binding moiety that binds E3 ubiquitin ligase selected from the group consisting of VHL, IAP, Cereblon, and mouse double minute 2 (MDM2). Preferably ULM includes but is not limited to, the following:

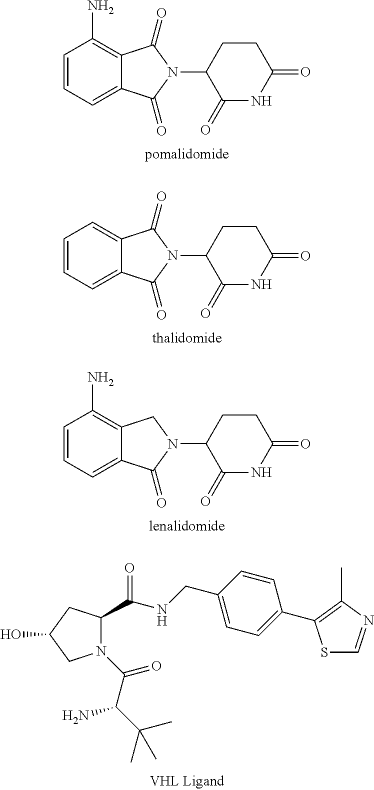 [SMILES: Nc1cccc2c1C(=O)N(C1CCC(=O)NC1=O)C2=O ; O=C1CCC(N2C(=O)c3ccccc3C2=O)C(=O)N1 ; Nc1cccc2c1CN(C1CCC(=O)NC1=O)C2=O ; Cc1ncsc1-c1ccc(CNC(=O)[C@@H]2C[C@@H](O)CN2C(=O)[C@@H](N)C(C)(C)C)cc1]

As used herein the term “PROTACs” represents proteolysis-targeting chimeras. Proteolysis-targeting chimeras (PROTACs) are heterobifunctional compounds with two recruiting ligands connected via a linker. One ligand is specific to the protein of interest (POI) while the other moiety specifically recruits an E3 ligase. The PROTAC thus forms a ternary complex upon binding to both its E3 ligase target and the protein of interest. By hijacking the E3 ligase, PROTACS position the POI in a spatially favorable presentation to facilitate substrate poly-ubiquitination, thereby selectively knocking down levels of the targeted protein (e.g., Crews, C. M., et al., Angewandte Chemie. Int. Ed. 55, 1966-1973, 2016).

The terms “optical isomer”, “geometric isomer” (e.g., a cis and/or trans isomer), “tautomers” (e.g., the tautomerism of keto form and enol form), “stereoisomer” and “diastereomer” have the accepted meanings (see, e.g., Hawley's Condensed Chemical Dictionary, 11^{th} Ed.). The illustration of specific protected forms and other derivatives of the compounds of the instant invention are not intended to be limiting. The application of other useful protecting groups, salt forms, prodrugs etc., is within the ability of the skilled artisan.

The term “prodrug” is a form of drug that must undergo chemical conversion by metabolic processes before becoming an active, or fully active, pharmacological agent. A prodrug is not active or is less active, in its ingested or absorbed or otherwise administered form. For example, a prodrug may be broken down by bacteria in the digestive system into products, at least one of which will become active as a drug. Alternatively, it may be administered systemically, such as by intravenous injection, and subsequently be metabolized into one or more active molecules.

The term “solvate” is a complex formed by the combination of a solute (e.g., a metalloprotease inhibitor) and a solvent (e.g., water). See J. Honig et al., The Van Nostrand Chemist's Dictionary, p. 650 (1953).

The term “independently” groups are groups present in the same structure that need not all represent the same substitution.

### DETAILED DESCRIPTION OF THE INVENTION

Experiments conducted during the course of developing embodiments for the present invention involved discovery of a series of 2-hydroxybenzoic acid derivatives as the inhibitors and degraders of sirtuin5 (SIRT5). Sirtuins are a class of nicotinamide adenine dinucleotide (NAD⁺)-dependent deacylases. There are seven sirtuin members encoded in mammalian genomes, SIRT1 through SIRT7. Unlike other sirtuin family members, SIRT5 is an inefficient deacetylase; instead it possesses very efficient desuccinylase, demalonylase, and deglutarylase activities, thereby influencing multiple cellular pathways such as ammonia detoxification, fatty acid oxidation, cellular respiration, ketone body formation, tricarboxylic acid cycle (TCA), glycolysis and reactive oxygen species (ROS) metabolism. Dysregulation of SIRT5 is found in several human diseases, especially cancer. It was found that SIRT5 depletion in specific cancer types (e.g., melanoma, Ewing sarcoma, malignant peripheral nerve sheath tumor, neuroblastoma) induces rapid cell death. Such experiments involved a screening an in-house library of compounds using a thermal shift assay and identified a novel class of 2-hydroxybenzoic acid derivatives as SIRT5 inhibitors. Such experiments also involved a successful development of PROTACs selectively inducing the degradation of SIRT5 protein that mainly resides in the mitochondrial matrix. These inhibitors and degraders are contemplated to useful in the treatment of disorders associated with sirtuins, for example, cancer (e.g., melanoma, Ewing sarcoma, malignant peripheral nerve sheath tumor, neuroblastoma, non-small cell lung cancer).

Accordingly, the present invention relates to a new class of small-molecules having a 2-hydroxybenzoic acid structure which function as sirtuin (e.g., SIRT1, SIRT2, SIRT3, SIRT4, SIRT5, SIRT6, SIRT7) inhibitors and/or degraders which function as effective therapeutic agents for treating, ameliorating, and preventing disorders associated with sirtuin activity (e.g., melanoma, Ewing sarcoma, malignant peripheral nerve sheath tumor, non-small cell lung cancer). In addition, this invention also relates to a new class of proteolysis-targeting chimeras (PROTACs) (as defined herein) which function as sirtuin inhibitors and/or degraders within cancer and/or immune cells. Pharmaceutical compositions comprising said compounds are also within the scope of the present invention.

In a particular embodiment, compounds encompassed within Formula (1), Formula (2), or Formula (3) are provided:

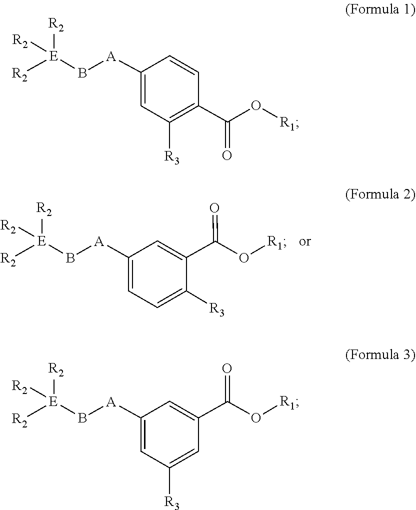

including pharmaceutically acceptable salts, solvates, PROTACs, and/or prodrugs thereof.

Formulas (1), (2), and (3) are not limited to a particular chemical moiety for R1, R2, R3, A, B and E. In some embodiments, the particular chemical moiety for R1, R2, R3, A, B and E independently include any chemical moiety that permits the resulting compound to inhibit and/or degrade sirtuin activity (e.g., SIRT1, SIRT2, SIRT3, SIRT4, SIRT5, SIRT6, SIRT7). In some embodiments, the particular chemical moiety for R1, R2, R3, A, B and E independently include any chemical moiety that permits the resulting compound to inhibit and/or degrade one or more of SIRT1, SIRT2, SIRT3, SIRT4, SIRT5, SIRT6, SIRT7. In some embodiments, the particular chemical moiety for R1, R2, R3, A, B and E independently include any chemical moiety that permits the resulting compound to inhibit and/or degrade SIRT5 activity. In some embodiments, the particular chemical moiety for R1, R2, R3, A, B and E independently include any chemical moiety that permits the resulting compound to inhibit and/or degrade sirtuin (e.g., SIRT5) related desuccinylase activity. In some embodiments, the particular chemical moiety for R1, R2, R3, A, B and E independently include any chemical moiety that permits the resulting compound to inhibit and/or degrade sirtuin (e.g., SIRT5) related demalonylase activity. In some embodiments, the particular chemical moiety for R1, R2, R3, A, B and E independently include any chemical moiety that permits the resulting compound to inhibit and/or degrade sirtuin (e.g., SIRT5) related deglutarylase activity. In some embodiments, the particular chemical moiety for R1, R2, R3, A, B and E independently include any chemical moiety that permits the resulting compound to influence multiple cellular pathways related to sirtuin activity (e.g., SIRT5) such as ammonia detoxification, fatty acid oxidation, cellular respiration, ketone body formation, tricarboxylic acid cycle (TCA), glycolysis and reactive oxygen species (ROS) metabolism. In some embodiments, the particular chemical moiety for R1, R2, R3, A, B and E independently include any chemical moiety that permits the resulting compound to serve as a therapeutic for treating, preventing and/or ameliorating disorders characterized with sirtuin (e.g., SIRT5) activity (e.g., sirtuin related cancer (e.g., melanoma, Ewing sarcoma, malignant peripheral nerve sheath tumor, non-small cell lung cancer).

In some embodiments, R¹ is selected from the group consisting of hydrogen, alkyl, cycloalkyl, heteroalkyl, heterocycloalkyl, aryl, heteroaryl, arylalkyl, and heteroarylalkyl.

In some embodiments, R¹ is selected from the group consisting of hydrogen, methyl, ethyl, n-propyl, isopropyl, n-butyl, isobutyl, sec-butyl, tert-butyl, pentyl,

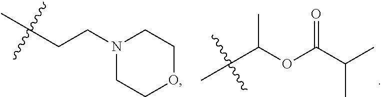

In some embodiments, A is selected from the group consisting of NH, CH₂, O, S,

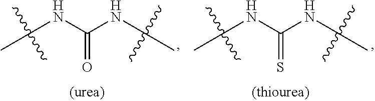 [SMILES: *NC(=O)N* ; *NC(=S)N*]

amide, and sulfonamide.

In some embodiments, B is an aromatic ring.

In some embodiments, B is a thiazole ring (e.g.,

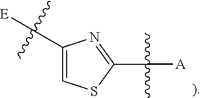

In some embodiments, E is an aromatic ring.

In some embodiments, E is a benzene ring.

In some embodiments,

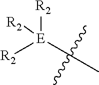

is selected from:

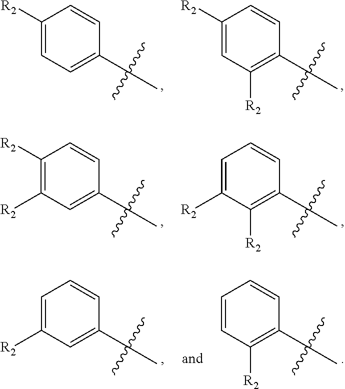

In some embodiments, each R² within

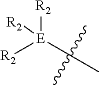

is independently selected from the group consisting of hydrogen, halogen, (e.g., F, Cl, Br, I), alkyl, cycloalkyl, heteroalkyl, heterocycloalkyl, halogen, aryl, heteroaryl, arylalkyl, heteroarylalkyl, nitryl, cyano, amide or sulfonamide,

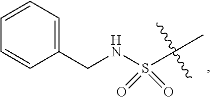 [SMILES: *S(=O)(=O)NCc1ccccc1]

and triphenylphosphine (TPP) attached with linker; and/or
wherein each R² within

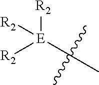

is independently selected from the group consisting of hydrogen, halogen, CF₃, OCH₃, OH, NO₂ (nitryl), CN (cyano), amide (e.g., NHC(O)CH3), and sulfonamide (e.g., S(O2)NHCH3),

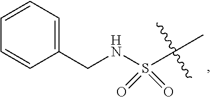 [SMILES: *S(=O)(=O)NCc1ccccc1]

and triphenylphosphine (TPP) group attached with a linker.

In some embodiments, linker is covalently bonded to connect two parts; the carbon atom in the linear chain can be substituted with oxygen, nitrogen, sulfur, ester, and amide.

In some embodiments, linker is of following Formula (L0):

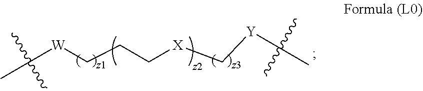

or an enantiomer, diastereomer, or stereoisomer thereof, wherein z1 is an integer selected from 0 to 10; Z2 is an integer selected from 0 to 10; Z3 is an integer selected from 0 to 10; each X is independently absent, CH₂, O, S, NH, NR₁₃; wherein W is selected from absent, O, NH, NR₁₃, —OCH₂C(O)NH—, —CH₂CH₂C(O)NH—, —CH₂C(O)NH— or —C(O)NH—; wherein Y is absent, O, NH, NR₁₃, —OCH₂C(O)NH—, —CH₂CH₂C(O)NH—, —CH₂C(O)NH— or —C(O)NH—; and each R1₃ is independently C₁-C₃ alkyl. The linker can be substituted with an alkyl, halide, phenyl, benzyl, aryl, alkylene or heterocycle group.

In some embodiments, linker is one of the following moieties:

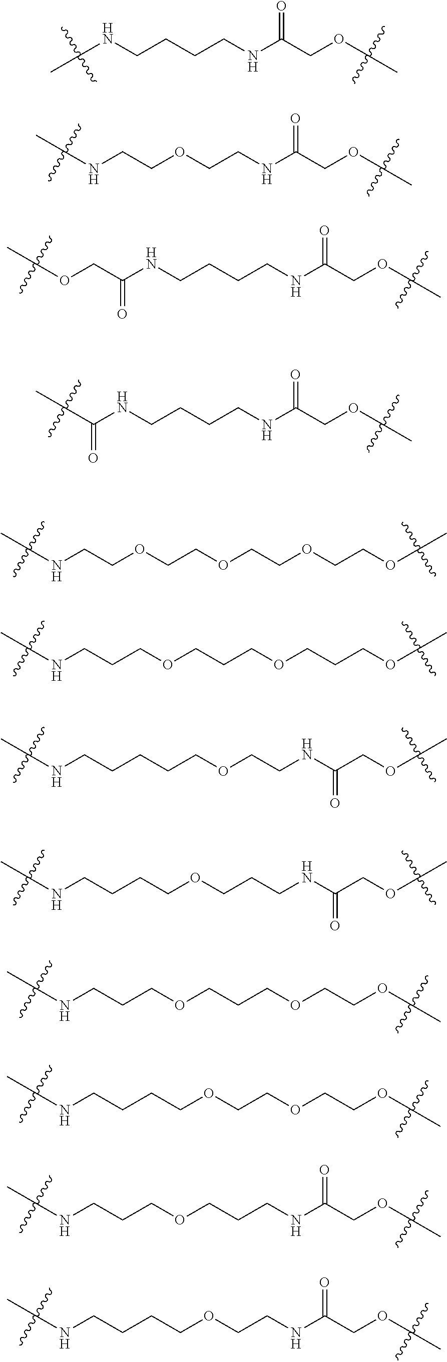

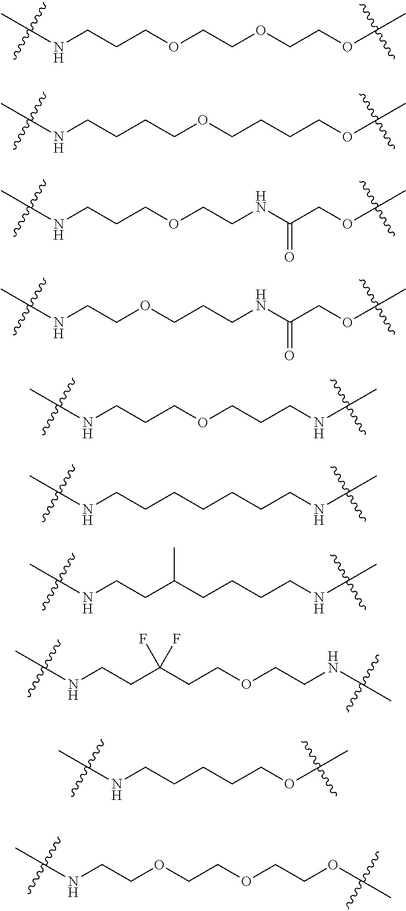

In some embodiments, R² is represented by Formula (P0):

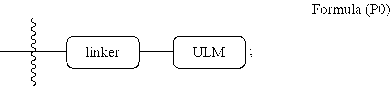

wherein the linker is covalently bonded to connect two parts; wherein the carbon atom in the linear chain can be substituted with oxygen, nitrogen, sulfur, ester, and amide; wherein ULM represents an E3 ubiquitin ligase binding moiety that binds E3 ubiquitin ligase selected from the group consisting of pomalidomide, thalidomide, lenalidomide, Von Hippel-Lindau (VHL), inhibitors of apoptosis proteins (IAP), Cereblon, and mouse double minute 2 (MDM2).

In some embodiments, R3 is hydrogen or OH.

In some embodiments, prodrug is preferably carboxylic acid ester. The ester group masks the negative charge on the carboxylate and thus can increase the cell permeability of the compounds. Inside the cells, the ester group can be hydrolyzed to release the negatively charged carboxylate, which can inhibit SIRT5.

In some embodiments, compounds shown in Table I are contemplated for Formula (1). Table I additionally shows values for SIRT5 IC₅₀ for select compounds.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, cancer associated with sirtuin (e.g., SIRT5) activity (e.g., melanoma, Ewing sarcoma, malignant peripheral nerve sheath tumor, non-small cell lung cancer).

A non-limiting exemplary list of cancers include, but are not limited to, pancreatic cancer, breast cancer, prostate cancer, lymphoma, skin cancer, colon cancer, melanoma, malignant melanoma, ovarian cancer, brain cancer, primary brain carcinoma, head and neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer cells being treated are metastatic. In other embodiments, the cancer cells being treated are resistant to anticancer agents.

Some embodiments of the present invention provide methods for administering an effective amount of a compound of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies).

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 4) antiestrogens (e.g., tamoxifen); 15) androgens (e.g., testosterone propionate and fluoxymesterone); 16) antiandrogens (e.g., flutamide); and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 2 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 2

| | | |
|---|---|---|
| Aldesleukin | Proleukin | Chiron Corp., |
| (des-alanyl-1,serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, |
| | | Inc., San Diego CA |
| Allopurinol | Zyloprim | GlaxoSmithKline, |
| (1,5-dihydro-4 H -pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol,2-[(3-aminopropyl)amino]-, | | |
| dihydrogen phosphate (ester)) | | |
| Anastrozole | Arimidex | AstraZeneca |
| (1,3-Benzenediacetonitrile,a,a,a′,a′-tetramethyl-5- | | Pharmaceuticals, |
| (1H-1,2,4-triazol-1-ylmethyl)) | | LP, Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand Pharmaceuticals |
| Bleomycin | Blenoxane | Bristol-Myers |
| (cytotoxic glycopeptide antibiotics produced by | | Squibb Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers |
| (platinum, diammine[1,1- | | Squibb |
| cyclobutanedicarboxylato(2-)-0,0′]-,(SP-4-2)) | | |
| Carmustine | BCNU, BiCNU | Bristol-Myers |
| (1,3-bis(2-chloroethyl)-1-nitrosourea) | | Squibb |
| Carmustine with Polifeprosan 20 Implant | Gliadel Wafer | Guilford |
| | | Pharmaceuticals, |
| | | Inc., Baltimore, MD |
| Celecoxib | Celebrex | Searle |
| (as 4-[5-(4-methylphenyl)-3-(trifluoromethyl)-1H- | | Pharmaceuticals, |
| pyrazol-1-yl]benzenesulfonamide) | | England |
| Chlorambucil | Leukeran | GlaxoSmithKline |
| (4-[bis(2chlorethyl)amino]benzenebutanoic acid) | | |
| Cisplatin | Platinol | Bristol-Myers |
| (PtCl₂H₆N₂) | | Squibb |
| Cladribine | Leustatin, 2-CdA | R.W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers |
| (2-┌bis(2-chloroethyl)amino┐tetrahydro-2H-13,2- | | Squibb |
| oxazaphosphorine 2-oxide monohydrate) | | |
| Cytarabine | Cytosar-U | Pharmacia & |
| (1-b-D-Arabinofuranosylcytosine, C₉H₁₃N₃O₅) | | Upjohn Company |
| cytarabine liposomal | DepoCyt | Skye |
| | | Pharmaceuticals, |
| | | Inc., San Diego, CA |
| Dacarbazine | DTIC-Dome | Bayer AG, |
| (5-(3,3-dimethyl-l-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-á-L- | | Pharmaceuticals, |
| lyxo-hexopyranosyl)oxy]-7,8,9,10-tetrahydro- | | Inc., Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1S,3S)-3-Acetyl-1,2,3,4,6,11-hexahydro-3,5,12- | | Madison, NJ |
| trihydroxy-10-methoxy-6,11-dioxo-1-naphthacenyl | | |
| 3-amino-2,3,6-trideoxy-(alpha)-L-lyxo- | | |
| hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & |
| ((S)-4,4′-(1-methyl-1,2-ethanediyl)bis-2,6- | | Upjohn Company |
| piperazinedione) | | |
| Docetaxel | Taxotere | Aventis |
| ((2R,3S)-N-carboxy-3-phenylisoserine,N-tert-butyl | | Pharmaceuticals, |
| ester, 13-ester with 5b-20-epoxy-12a,4,7b,10b,13a- | | Inc., Bridgewater, |
| hexahydroxytax-11-en-9-one 4-acetate 2-benzoate, | | NJ |
| trihydrate) | | |
| Doxorubicin HCl | Adriamycin, | Pharmacia & |
| (8S,10S)-10-[(3-amino-2,3,6-trideoxy-a-L-lyxo- | Rubex | Upjohn Company |
| hexopyranosyl)oxy]-8-glycolyl-7,8,9,10-tetrahydro- | | |
| 6,8,11-trihydroxy-1-methoxy-5,12- | | |
| naphthacenedione hydrochloride) | | |
| doxorubicin | Adriamycin PFS | Pharmacia & |
| | Intravenous | Upjohn Company |
| | injection | |
| doxorubicin liposomal | Doxil | Sequus |
| | | Pharmaceuticals, |
| | | Inc., Menlo park, CA |
| dromostanolone propionate | Dromostanolone | Eli Lilly & |
| (17b-Hydroxy-2a-methyl-5a-androstan-3-one | | Company, |
| propionate) | | Indianapolis, IN |
| dromostanolone propionate | Masterone | Syntex, Corp., Palo |
| | injection | Alto, CA |
| Elliott′s B Solution | Elliott's B | Orphan Medical, Inc |
| | Solution | |
| Epirubicin | Ellence | Pharmacia & |
| ((8S-cis)-10-[(3-amino-2,3,6-trideoxy-a-L-arabino- | | Upjohn Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & |
| (estra-1,3,5(10)-triene-3,17-diol(17(beta))-,3-[bis(2- | | Upjohn Company |
| chloroethyl)carbamate┐ 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers |
| (4′-Demethylepipodophyllotoxin 9-[4,6-O-(R)- | | Squibb |
| ethylidene-(beta)-D-glucopyranoside],4′- | | |
| (dihydrogen phosphate)) | | |
| etoposide, VP-16 | Vepesid | Bristol-Myers |
| (4′-demethylepipodophyllotoxin 9-[4,6-0-(R)- | | Squibb |
| ethylidene-(beta)-D-glucopyranoside]) | | |
| Exemestane | Aromasin | Pharmacia & |
| (6-methylenandrosta-1,4-diene-3,17-dione) | | Upjohn Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral agent | | Inc., Cedar Knolls, NJ |
| vidarabine, 9-b-D-arabinofuranosyladenine (ara-A)) | | |
| Fluorouracil, 5-FU | Adrucil | ICN |
| (5-fluoro-2,4(1H,3H)-pyrimidinedione) | | Pharmaceuticals, |
| | | Inc., Humacao, |
| | | Puerto Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine monohydrochloride | | |
| (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| | | Pharmaceuticals |
| Hydroxyurea | Hydrea | Bristol-Myers |
| | | Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & |
| (5,12-Naphthacenedione,9-acetyl-7-[(3-amino- | | Upjohn Company |
| 2,3,6-trideoxy-(alpha)-L-lyxo-hexopyranosyl)oxy]- | | |
| 7,8,9,10-tetrahydro-6,9,11-trihydroxyhydrochloride, | | |
| (7S- cis)) | | |
| Ifosfamide | IFEX | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl-3- | | Switzerland |
| [[4-(3-pyridinyl)-2-pyrimidinyl]amino]- | | |
| phenyl]benzamide methanesulfonate) | | |
| Interferon alfa-2a | Roferon-A | Hoffmann-La |
| (recombinant peptide) | | Roche, Inc., Nutley, |
| | | NJ |
| Interferon alfa-2b | Intron A | Schering AG, |
| (recombinant peptide) | (Lyophilized | Berlin, Germany |
| | Betaseron) | |
| Irinotecan HCl | Camptosar | Pharmacia & |
| ((4S)-4,11-diethyl-4-hydroxy-9-[(4-piperi- | | Upjohn Company |
| dinopiperidino)carbonyloxy]-1H-pyrano[3′,4′: 6,7] | | |
| indolizino[1,2-b] quinoline-3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl1,4,5,6,7,8 | Leucovorin | Seattle, WA |
| hexahydro4oxo6-pteridinyl)methyl]amino]benzoyl], | | |
| calcium salt (1:1)) | | |
| Levamisole HCl | Ergamisol | Janssen Research |
| ((—)-(S)-2,3,5,6-tetrahydro-6-phenylimidazo [2,1-b] | | Foundation, |
| thiazole monohydrochloride C₁₁H₁₂N₂S•HCl) | | Titusville, NJ |
| Lomustine | CeeNU | Bristol-Myers |
| (1-(2-chloro-ethyl)-3-cyclohexyl-1-nitrosourea) | | Squibb |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers |
| 17α(acetyloxy)-6-methylpregna-4,6-diene-3,20-dione | | Squibb |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle |
| (N-[4-[[(2,4-diamino-6- | | Laboratories |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers |
| | | Squibb |
| mitomycin C | Mitozytrex | SuperGen, Inc., |
| | | Dublin, CA |
| Mitotane | Lysodren | Bristol-Myers |
| (1,1-dichloro-2-(o-chlorophenyl)-2-(p-chlorophenyl) | | Squibb |
| ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer |
| | | Ingelheim Pharma |
| | | KG, Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers |
| (5β,20-Epoxy-1,2a,4,7β,10β,13a-hexahydroxytax- | | Squibb |
| 11-en-9-one 4,10-diacetate 2-benzoate 13-ester with | | |
| (2R,3S)-N-benzoyl-3-phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, |
| 11-17-adenosine deaminase) | Bovine) | Inc., Bridgewater, |
| | | NJ |
| Pegaspargase | Oncaspar | Enzon |
| (monomethoxypolyethylene glycol succinimidyl L- | | |
| asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott |
| | | Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San |
| | | Francisco, CA |
| Sargramostim | Prokine | Immunex Corp |
| (recombinant peptide) | | |
| Streptozocin | Zanosar | Pharmacia & |
| (streptozocin 2-deoxy-2- | | Upjohn Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀ (OH)₂) | | Woburn, MA |
| Tamoxifen | Nolvadex | AstraZeneca |
| ((Z)2-[4-(1,2-diphenyl-1-butenyl)phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers |
| (4′-demethylepipodophyllotoxin 9-[4,6-0-(R)-2- | | Squibb |
| thenylidene-(beta)-D-glucopyranoside]) | | |
| Testolactone | Teslac | Bristol-Myers |
| (13-hydroxy-3-oxo-13,17-secoandrosta-1,4-dien-17- | | Squibb |
| oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex |
| (Aziridine,1,1′,1″-phosphinothioylidynetris-, or Tris | | Corporation |
| (1-aziridinyl) phosphine sulfide) | | |
| Topotecan HCl | Hycamtin | GlaxoSmithKline |
| ((S)-10-[(dimethylamino)methyl]-4-ethyl-4,9- | | |
| dihydroxy-1H-pyrano[3′,4′: 6,7] indolizino [1,2-b] | | |
| quinoline-3,14-(4H,12H)-dione monohydrochloride) | | |
| Toremifene | Fareston | Roberts |
| (2-(p-[(Z)-4-chloro-1,2-diphenyl-1-butenyl]- | | Pharmaceutical Corp., |
| phenoxy)-N,N-dimethylethylamine citrate (1:1)) | | Eatontown, NJ |
| Tositumomab, I 131 Tositumomab | Bexxar | Corixa Corp., |
| (recombinant murine immunotherapeutic | | Seattle, WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14-valerate | Valstar | Anthra --> Medeva |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′-norvincaleukoblastine | | |
| [R-(R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, 1BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflormithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafamib, luniliximab, mafosfamide, M1B07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

The present invention provides methods for administering a compound of the invention with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to an animal is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. As used herein, the use of personal pronouns such as “we”, “I”, and/or “our” refers to the inventors.

### Example 1

### Preparation of 4-((4-(4-acetamidophenyl)thiazol-2-yl)amino)-2-hydroxybenzoic acid (Compound 1-1)

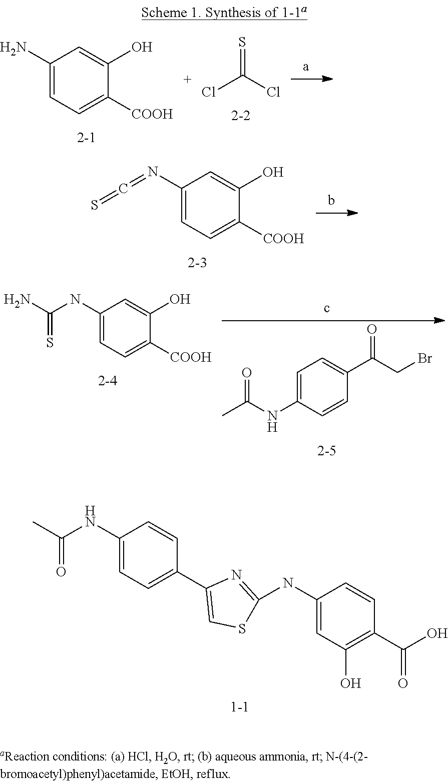

2-hydroxy-4-isothiocyanatobenzoic acid (2-3). 4-Aminosalicylic acid (3.06 g, 0.02 mol) was suspended in 35 mL of water and 6.7 mL of concentrated hydrochloric acid added with mechanical stirring. To this suspension another 35 mL portion of water was added and finally thiophosgene (2.7 g, 0.024 mol) was added in one portion. After 3 hours of stirring, the orange color of the thiophosgene was dissipated indicating completion of the reaction. The solid product was filtered; the filter cake was washed several times with water and dried over phosphorus pentoxide. This product (3.35 g, 86% yield) of white solid that was recrystallized from toluene. The pure product was slightly unstable to light. ¹H NMR (400 MHz, DMSO-d₆) δ 7.83 (d, J=8.4 Hz, 1H), 7.00 (d, J=2.0 Hz, 1H), 6.96 (dd, J=8.4, 2.1 Hz, 1H).

2-hydroxy-4-thioureidobenzoic acid (2-4). Compound 2-3 (3.756 g, 0.019 mol) was dissolved in 70 mL of aqueous ammonia (28%) and the solution stirred overnight. This product (2.98 g, 73% yield) of white solid was recrystallized from aqueous ethanol. ¹H NMR (400 MHz, DMSO-d₆) δ 11.38 (s, 1H), 9.97 (s, 1H), 8.07 (s, 1H), 7.70 (d, J=8.7 Hz, 1H), 7.38 (d, J=2.1 Hz, 1H), 6.96 (dd, J=8.7, 2.2 Hz, 1H).

4-((4-(4-acetamidophenyl)thiazol-2-yl)amino)-2-hydroxybenzoic acid (1-1). To a solution of compound 2-4 (424 mg, 2.0 mmol) in ethanol was added N-(4-(2-bromoacetyl)phenyl)acetamide 2-5 (512 mg, 2.0 mmol). The reaction mixture was refluxed for 3 h. Preparative HPLC purification afforded the pure product 1-1 as a white solid (400 mg, 54% yield). ¹H NMR (300 MHz, DMSO-d₆) δ 10.72 (s, 1H), 10.10 (s, 1H), 7.84 (d, J=8.5 Hz, 2H), 7.74 (d, J=8.7 Hz, 1H), 7.67 (d, J=8.5 Hz, 2H), 7.56 (d, J=2.1 Hz, 1H), 7.34 (s, 1H), 7.10 (dd, J=8.9, 2.1 Hz, 1H), 2.07 (s, 3H). LCMS (ESI) 370 [M+H]⁺, 368 [M−H]⁻. HPLC purity at 254 nm, 99.5%.

### Example 2

### Preparation of ethyl4-((4-(4-acetamidophenyl)thiazol-2-yl)amino)-2-hydroxybenzoate (Compound 1-2)

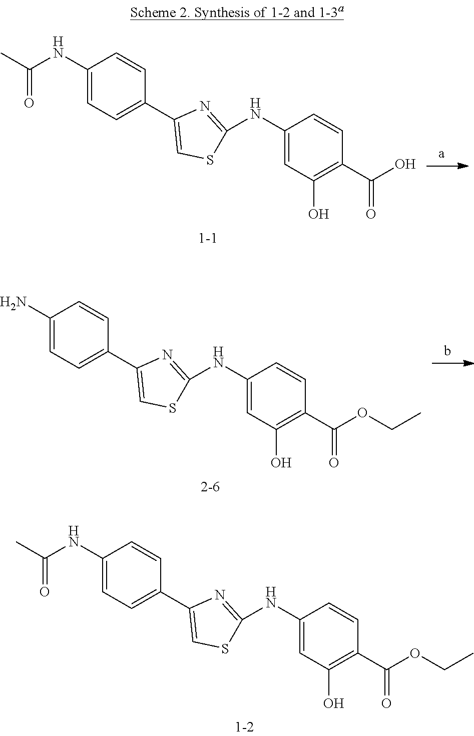

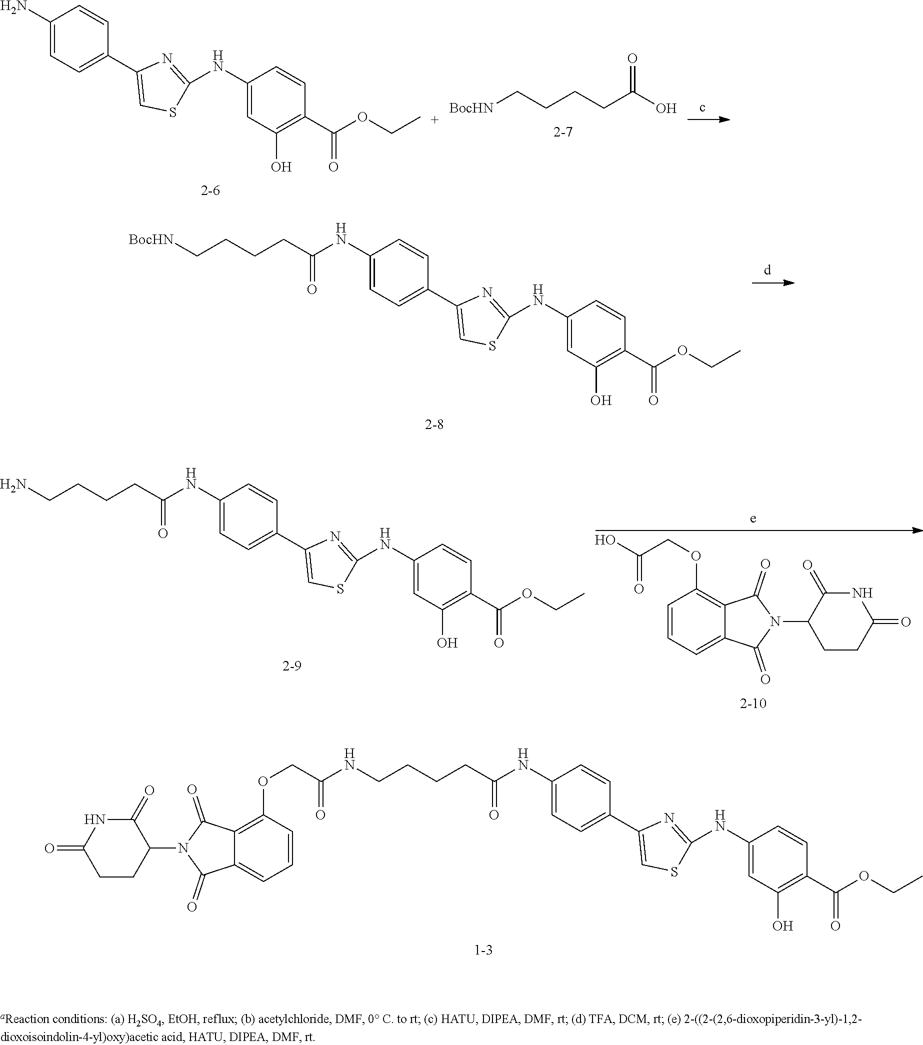

Ethyl 4-((4-(4-aminophenyl)thiazol-2-yl)amino)-2-hydroxybenzoate (2-6). To a solution of compound 1-1 (370 mg, 1 mmol) in ethanol (3 mL) was added conc. H₂SO₄ (0.2 mL). The reaction mixture was refluxed for 8 h. Preparative HPLC purification afforded the pure product 2-6 as a white solid (200 mg, 56% yield).

¹H NMR (400 MHz, DMSO-d₆) δ 10.88 (s, 1H), 10.70 (s, 1H), 7.84-7.80 (m, 2H), 7.77 (d, J=8.8 Hz, 1H), 7.59 (d, J=2.1 Hz, 1H), 7.30 (s, 1H), 7.12 (dd, J=8.8, 2.2 Hz, 1H), 7.02 (d, J=8.5 Hz, 2H), 4.35 (q, J=7.1 Hz, 2H), 1.35 (t, J=7.1 Hz, 3H). MS (ESI) 356 [M+H]⁺.

Ethyl 4-((4-(4-acetamidophenyl)thiazol-2-yl)amino)-2-hydroxybenzoate (1-2). Compound 2-6 (30 mg, 0.085 mmol) was dissolved in anhydrous DMF (2 mL). Acetylchloride (10 μL, 0.141 mmol) and DIPEA (40 μL, 0.242 mmol) were added sequentially at 0° C. The reaction mixture was then allowed to reach ambient temperature and stirred for 2 h. Preparative HPLC purification afforded the pure product 1-2 as a white solid (29 mg, 86% yield). ¹H NMR (400 MHz, DMSO-d₆) δ 10.87 (s, 1H), 10.71 (s, 1H), 10.06 (s, 1H), 7.86 (d, J=8.7 Hz, 2H), 7.78 (d, J=8.8 Hz, 1H), 7.67 (d, J=8.8 Hz, 2H), 7.57 (d, J=2.1 Hz, 1H), 7.36 (s, 1H), 7.15 (dd, J=8.8, 2.2 Hz, 1H), 4.35 (q, J=7.1 Hz, 2H), 2.07 (s, 3H), 1.35 (t, J=7.1 Hz, 3H). LCMS (ESI) 398 [M+H]⁺, 396 [M−H]⁻. HPLC purity at 254 nm, 99.6%.

### Example 3

### Preparation of ethyl 4-((4-(4-(5-(2-((2-(2,6-dioxopiperidin-3-yl)-1,3-dioxoisoindolin-4-yl)oxy)acetamido)pentanamido)phenyl)thiazol-2-yl)amino)-2-hydroxybenzoate (compound 1-3)

Ethyl 4-((4-(4-(5-((tert-butoxycarbonyl)amino)pentanamido)phenyl)thiazol-2-yl)-amino)-2-hydroxybenzoate (2-8). Compound 2-6 (35 mg, 0.1 mmol) was dissolved in anhydrous DMF (2 mL). 5-((tert-butoxycarbonyl)amino)pentanoic acid 2-7 (22 mg, 0.1 mmol), HATU (42 mg, 0.11 mmol), and DIPEA (50 μL, 0.3 mmol) were added sequentially. The reaction mixture was stirred at room temperature for 2 h and then purified by HPLC to isolate the product as a slightly yellow solid (40 mg, 72% yield). ¹H NMR (400 MHz, DMSO-d₆) δ 10.87 (s, 1H), 10.71 (s, 1H), 9.99 (s, 1H), 7.89-7.82 (m, 2H), 7.78 (d, J=8.8 Hz, 1H), 7.68 (d, J=8.8 Hz, 2H), 7.56 (d, J=2.1 Hz, 1H), 7.37 (s, 1H), 7.15 (dd, J=8.8, 2.2 Hz, 1H), 6.83 (t, J=5.6 Hz, 1H), 4.35 (q, J=7.1 Hz, 2H), 2.95 (q, J=6.7 Hz, 2H), 2.33 (t, J=7.3 Hz, 2H), 1.59 (p, J=7.6 Hz, 2H), 1.43 (q, J=7.5 Hz, 2H), 1.38 (s, 9H), 1.35 (t, J=7.1 Hz, 3H). MS (ESI) 555 [M+H]⁺.

Intermediate 2-8 (30 mg, 0.054 mmol) was dissolved in DCM (2 mL) and TFA (1 mL). After stirring for 2 h, the solvent was evaporated and the residue 2-9 was used in the next step without further purification.

Ethyl 4-((4-(4-(5-(2-((2-(2,6-dioxopiperidin-3-yl)-1,3-dioxoisoindolin-4-yl)oxy)-acetamido)pentanamido)phenyl)thiazol-2-yl)amino)-2-hydroxybenzoate (1-3). The amine intermediate 2-9 (25 mg, 0.055 mmol), HATU (25 mg, 0.065 mmol) and DIPEA (30 μL, 0.18 mmol) were added sequentially to a stirred solution of compound 2-((2-(2,6-dioxopiperidin-3-yl)-1,3-dioxoisoindolin-4-yl)oxy)acetic acid 2-10 (18 mg, 0.055 mmol) in DMF (2 mL). The reaction mixture was stirred at room temperature for 2 h and then purified by HPLC to get the product as a white solid (32 mg, 75% yield). ¹H NMR (400 MHz, DMSO-d₆) δ 11.13 (s, 1H), 10.88 (s, 1H), 10.72 (s, 1H), 10.01 (s, 1H), 8.03 (t, J=5.7 Hz, 1H), 7.89-7.75 (m, 4H), 7.68 (d, J=8.5 Hz, 2H), 7.57 (d, J=2.1 Hz, 1H), 7.49 (d, J=7.2 Hz, 1H), 7.41 (d, J=8.6 Hz, 1H), 7.36 (s, 1H), 7.15 (dd, J=8.8, 2.1 Hz, 1H), 5.13 (dd, J=12.9, 5.4 Hz, 1H), 4.80 (s, 2H), 4.35 (q, J=7.1 Hz, 2H), 3.20 (q, J=6.5 Hz, 2H), 2.90 (ddd, J=18.0, 14.0, 5.5 Hz, 1H), 2.64-2.55 (m, 2H), 2.36 (t, J=7.2 Hz, 2H), 2.05 (ddt, J=12.0, 7.0, 3.9 Hz, 1H), 1.63 (dq, J=12.5, 7.2 Hz, 2H), 1.51 (p, J=6.9 Hz, 2H), 1.35 (t, J=7.1 Hz, 3H). LCMS (ESI) 769 [M+H]⁺, 767 [M−H]⁻. HPLC purity at 254 nm, 99.5%.

### Example 4

### Preparation of ethyl 4-((4-(4-(1-((2-(2,6-dioxopiperidin-3-yl)-1,3-dioxoisoindolin-4-yl)oxy)-2-oxo-6,9,12,15,18-pentaoxa-3-azahenicosan-21-amido)phenyl)thiazol-2-yl)amino)-2-hydroxybenzoate (compound 1-4)

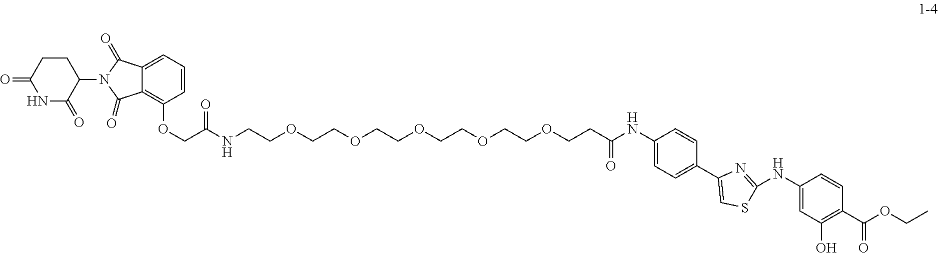 [SMILES: CCOC(=O)c1ccc(Nc2nc(-c3ccc(NC(=O)CCOCCOCCOCCOCCOCCNC(=O)COc4cccc5c4C(=O)N(C4CCC(=O)NC4=O)C5=O)cc3)cs2)cc1O]

Following similar procedure used to prepare compound 1-3, we synthesized compound 1-4. ¹H NMR (400 MHz, DMSO-d₆) δ 11.13 (s, 1H), 10.87 (s, 1H), 10.70 (s, 1H), 10.06 (s, 1H), 8.01 (t, J=5.7 Hz, 1H), 7.90-7.75 (m, 4H), 7.71-7.64 (m, 2H), 7.56 (d, J=2.1 Hz, 1H), 7.50 (d, J=7.2 Hz, 1H), 7.40 (dd, J=8.6, 0.6 Hz, 1H), 7.36 (s, 1H), 7.14 (dd, J=8.8, 2.2 Hz, 1H), 5.12 (dd, J=12.9, 5.4 Hz, 1H), 4.79 (s, 2H), 4.35 (q, J=7.1 Hz, 2H), 3.72 (t, J=6.2 Hz, 2H), 3.56-3.42 (m, 18H), 3.31 (q, J=5.7 Hz, 2H), 2.90 (ddd, J=17.3, 14.0, 5.4 Hz, 1H), 2.65-2.54 (m, 4H), 2.10-1.98 (m, 1H), 1.35 (t, J=7.1 Hz, 3H). LCMS (ESI) 961 [M+H]⁺, 959 [M−H]⁻. HPLC purity at 254 nm, 99.5%.

### Example 5

### Preparation of 4-(3-(4-(4-acetamidophenyl)thiazol-2-yl)thioureido)-2-hydroxybenzoic acid (Compound 1-5)

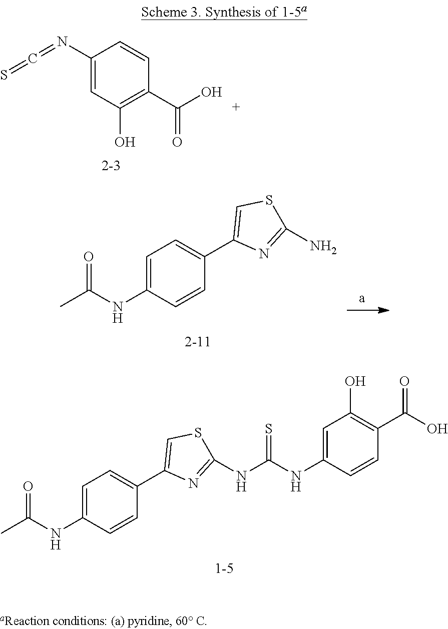 [SMILES: O=C(O)c1ccc(N=C=S)cc1O ; CC(=O)Nc1ccc(-c2csc(N)n2)cc1 ; CC(=O)Nc1ccc(-c2csc(NC(=S)Nc3ccc(C(=O)O)c(O)c3)n2)cc1]

### 4-(3-(4-(4-acetamidophenyl)thiazol-2-yl)thioureido)-2-hydroxybenzoic acid (1-5)

Intermediate 2-3 (20 mg, 0.1 mmol) and amine 2-11 (23 mg, 0.1 mmol) were dissolved in anhydrous pyridine (2 mL). The reaction mixture was stirred at 60° C. overnight and then purified by HPLC to get the product as a slightly yellow solid (26 mg, 60% yield). ¹H NMR (400 MHz, DMSO-d₆) δ 11.40 (s, 1H), 10.08 (s, 1H), 7.99-7.03 (m, 8H), 2.08 (d, J=2.4 Hz, 3H). LCMS (ESI) 429 [M+H]⁺, 427 [M−H]⁻. HPLC purity at 254 nm, 99.5%.

### Example 6

### Preparation of ethyl 4-(3-(4-(4-acetamidophenyl)thiazol-2-yl)thioureido)-2-hydroxybenzoate (compound 1-6)

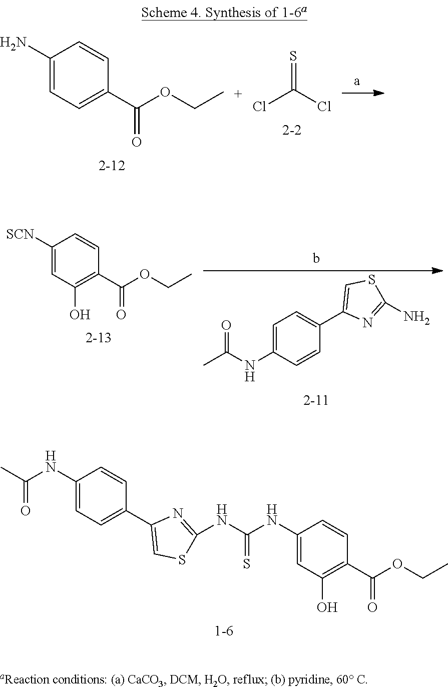

Ethyl 2-hydroxy-4-isothiocyanatobenzoate (2-13). A mixture of thiophosgene (228 μL, 3 mmol), CaCO₃ (175 mg, 1.75 mmol), DCM (2 mL) and H₂O (4 mL) was stirred at rt, then a solution of amine 2-12 (181 mg, 1 mmol) in DCM (2 mL) was added dropwise. The reaction mixture was refluxed for 48 h. The solid was suction-filtered and washed with CHCl₃ many times, the resulting mixture was extracted with CHCl₃, then the organic layer was washed with saturated aqueous NaCl, the residue was purified by flash silica chromatography to give this product as a slightly yellow solid (90 mg, 40% yield). ¹H NMR (400 MHz, Chloroform-d) δ 11.00 (s, 1H), 7.85 (dd, J=8.5, 0.4 Hz, 1H), 6.86-6.81 (m, 1H), 6.75 (dd, J=8.5, 2.0 Hz, 1H), 4.44 (q, J=7.1 Hz, 2H), 1.44 (t, J=7.2 Hz, 3H).

Ethyl 4-(3-(4-(4-acetamidophenyl)thiazol-2-yl)thioureido)-2-hydroxybenzoate (1-6). Intermediate ethyl 2-hydroxy-4-isothiocyanatobenzoate 2-13 (22 mg, 0.1 mmol) and amine 2-11 (23 mg, 0.1 mmol) were dissolved in anhydrous pyridine (2 mL). The reaction mixture was stirred at 60° C. overnight and then purified by HPLC to obtain the product as a white solid (27 mg, 60% yield). ¹H NMR (400 MHz, DMSO-d₆) δ 10.75 (s, 1H), 10.10 (s, 1H), 7.85-7.23 (m, 8H), 4.36 (q, J=7.1 Hz, 2H), 2.08 (s, 3H), 1.35 (t, J=7.1 Hz, 3H). LCMS (ESI) 457 [M+H]⁺, 455 [M−H]⁻. HPLC purity at 254 nm, 99.2%.

### Example 7

### Preparation of 4-(3-(4-(4-acetamidophenyl)thiazol-2-yl)thioureido)benzoic acid (Compound 1-7)

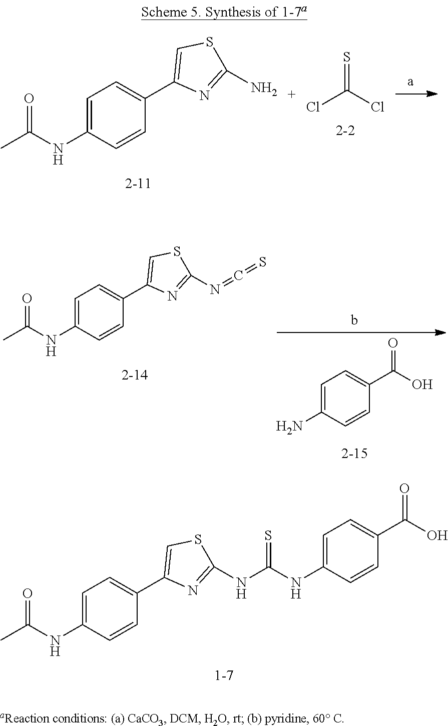

N-(4-(2-isothiocyanatothiazol-4-yl)phenyl)acetamide (2-14). A mixture of thiophosgene (114 μL, 1.5 mmol), CaCO₃ (175 mg, 1.75 mmol), DCM (2 mL) and H₂O (4 mL) was stirred at 0° C., then a solution of amine 2-11 (233 mg, 1 mmol) in DCM (2 mL) was added dropwise. The reaction mixture was stirred at room temperature overnight. The solid was suction-filtered and washed with CHCl₃ many times, the resulting mixture was extracted with CHCl₃, then the organic layer was washed with saturated aqueous NaCl, the residue was purified by flash silica chromatography to obtain the product as a slightly yellow solid (22 mg, 8% yield). ¹H NMR (400 MHz, Chloroform-d) δ 7.84-7.78 (m, 2H), 7.59 (d, J=8.5 Hz, 2H), 7.32 (s, 1H), 7.26 (s, 1H), 2.22 (s, 3H). MS (ESI) 276 [M+H]⁺.

4-(3-(4-(4-acetamidophenyl)thiazol-2-yl)thioureido)benzoic acid (1-7). Intermediate 2-14 (28 mg, 0.1 mmol) and amine 2-15 (14 mg, 0.1 mmol) were dissolved in anhydrous pyridine (2 mL). The reaction mixture was stirred at 60° C. overnight and then purified by HPLC to obtain the product as a white solid (26 mg, 62% yield). ¹H NMR (400 MHz, DMSO-d₆) δ 10.09 (s, 1H), 7.92 (q, J=8.7 Hz, 4H), 7.84-7.77 (m, 2H), 7.68 (d, J=8.7 Hz, 2H), 7.39 (s, 1H), 2.08 (s, 3H). LCMS (ESI) 413 [M+H]⁺, 411 [M−H]⁻. HPLC purity at 254 nm, 99.5%.

### Example 8

### Preparation of 5-(3-(4-(4-acetamidophenyl)thiazol-2-yl)thioureido)-2-hydroxybenzoic acid (Compound 1-8)

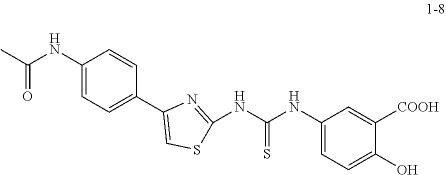 [SMILES: CC(=O)Nc1ccc(-c2csc(NC(=S)Nc3ccc(O)c(C(=O)O)c3)n2)cc1]

Compound 1-8 was obtained following the procedure used to prepare compound 1-7. ¹H NMR (400 MHz, DMSO-d₆) δ 11.21 (s, 1H), 10.72 (s, 1H), 10.05 (s, 1H), 8.08-7.30 (m, 7H), 6.96 (m, 1H), 2.07 (s, 3H). LCMS (ESI) 429 [M+H]⁺, 427 [M−H]⁻. HPLC purity at 254 nm, 97.1%.

### Example 9

### Preparation of 4-(3-(4-(4-acetamidophenyl)thiazol-2-yl)thioureido)-3-hydroxybenzoic acid (Compound 1-9)

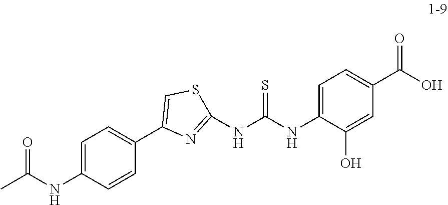 [SMILES: CC(=O)Nc1ccc(-c2csc(NC(=S)Nc3ccc(C(=O)O)cc3O)n2)cc1]

Compound 1-9 was obtained following the procedure used to prepare compound 1-7. ¹H NMR (400 MHz, DMSO-d₆) δ 12.79 (s, 1H), 12.22 (s, 1H), 10.78 (s, 1H), 10.06 (s, 1H), 8.72 (s, 1H), 8.05-7.18 (m, 8H), 2.08 (s, 3H). LCMS (ESI) 429 [M+H]⁺, 427 [M−H]⁻. HPLC purity at 254 nm, 99.7%.

### Example 10

### Preparation of 2-hydroxy-4-(3-(4-(p-tolyl)thiazol-2-yl)thioureido)benzoic acid (Compound 1-10)

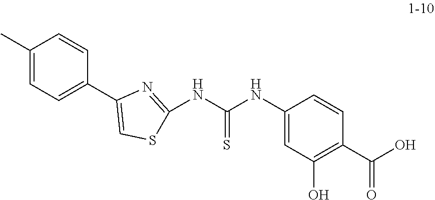 [SMILES: Cc1ccc(-c2csc(NC(=S)Nc3ccc(C(=O)O)c(O)c3)n2)cc1]

Compound 1-10 was obtained following the procedure used to prepare compound 1-5. ¹H NMR (400 MHz, DMSO-d₆) δ 7.81-7.60 (m, 4H), 7.46 (d, J=2.1 Hz, 1H), 7.29 (t, J=7.5 Hz, 3H), 2.34 (s, 3H). LCMS (ESI) 386 [M+H]⁺, 384 [M−H]⁻. HPLC purity at 254 nm, 99.5%.

### Example 11

### Preparation of 2-hydroxy-4-(3-(4-(m-tolyl)thiazol-2-yl)thioureido)benzoic acid (Compound 1-11)

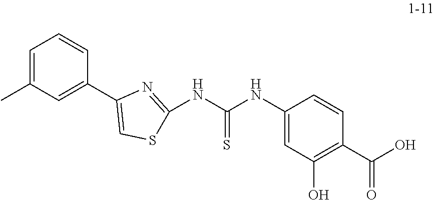 [SMILES: Cc1cccc(-c2csc(NC(=S)Nc3ccc(C(=O)O)c(O)c3)n2)c1]

Compound 1-11 was obtained following the procedure used to prepare compound 1-5. ¹H NMR (400 MHz, DMSO-d₆) δ 7.83-7.02 (m, 8H), 2.37 (s, 3H). LCMS (ESI) 386 [M+H]⁺, 384 [M−H]⁻. HPLC purity at 254 nm, 99.5%.

### Example 12

### Preparation of 2-hydroxy-4-(3-(4-(o-tolyl)thiazol-2-yl)thioureido)benzoic acid (Compound 1-12)

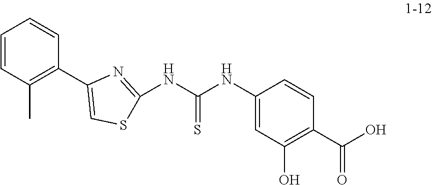 [SMILES: Cc1ccccc1-c1csc(NC(=S)Nc2ccc(C(=O)O)c(O)c2)n1]

Compound 1-12 was obtained following the procedure used to prepare compound 1-5. ¹H NMR (400 MHz, DMSO-d₆) δ 11.37 (s, 1H), 7.73-7.60 (m, 2H), 7.53-7.45 (m, 1H), 7.40-7.24 (m, 4H), 7.07 (s, 1H), 2.38 (s, 3H). LCMS (ESI) 386 [M+H]⁺, 384 [M−H]⁻. HPLC purity at 254 nm, 99.9%.

### Example 13

### Preparation of 4-(3-(4-(4-fluorophenyl)thiazol-2-yl)thioureido)-2-hydroxybenzoic acid (Compound 1-13)

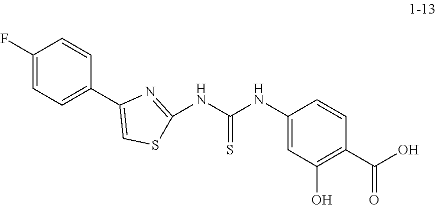 [SMILES: O=C(O)c1ccc(NC(=S)Nc2nc(-c3ccc(F)cc3)cs2)cc1O]

Compound 1-13 was obtained following the procedure used to prepare compound 1-5. ¹H NMR (400 MHz, DMSO-d₆) δ 11.41 (s, 1H), 10.80 (s, 1H), 7.93 (ddd, J=9.1, 5.2, 2.2 Hz, 2H), 7.74 (dd, J=18.1, 8.6 Hz, 1H), 7.55 (m, 2H), 7.42-7.15 (m, 3H). LCMS (ESI) 390 [M+H]⁺, 388 [M−H]⁻. HPLC purity at 254 nm, 99.9%.

### Example 14

### Preparation of 4-(3-(4-(3-fluorophenyl)thiazol-2-yl)thioureido)-2-hydroxybenzoic acid (Compound 1-14)

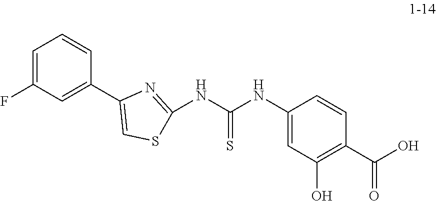 [SMILES: O=C(O)c1ccc(NC(=S)Nc2nc(-c3cccc(F)c3)cs2)cc1O]

Compound 1-14 was obtained following the procedure used to prepare compound 1-5. ¹H NMR (400 MHz, DMSO-d₆) δ 12.02 (s, 1H), δ 11.43 (s, 1H), 10.73 (s, 1H), δ 7.75 (q, J=11.0, 9.9 Hz, 4H), 7.63-7.42 (m, 2H), 7.21 (d, J=9.8 Hz, 2H). LCMS (ESI) 390 [M+H]⁺, 388 [M−H]⁻. HPLC purity at 254 nm, 99.4%.

### Example 15

### Preparation of 4-(3-(4-(2-fluorophenyl)thiazol-2-yl)thioureido)-2-hydroxybenzoic acid (Compound 1-15)

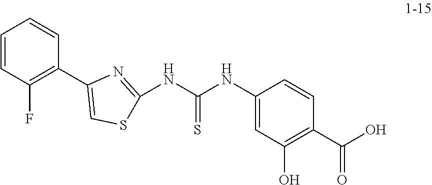 [SMILES: O=C(O)c1ccc(NC(=S)Nc2nc(-c3ccccc3F)cs2)cc1O]

Compound 1-15 was obtained following the procedure used to prepare compound 1-5. ¹H NMR (400 MHz, DMSO-d₆) δ 12.05 (s, 1H), S 11.41 (s, 1H), 10.89 (s, 1H), 7.99 (s, 1H), 7.75 (dd, J=17.9, 8.8 Hz, 1H), 7.63 (d, J=2.1 Hz, 1H), 7.54-7.30 (m, 4H), 7.20 (s, 1H). LCMS (ESI) 390 [M+H]⁺, 388 [M−H]⁻. HPLC purity at 254 nm, 99.9%.

### Example 16

### Preparation of 2-hydroxy-4-(3-(4-(4-(trifluoromethyl)phenyl)thiazol-2-yl)thioureido)benzoic acid (Compound 1-16)

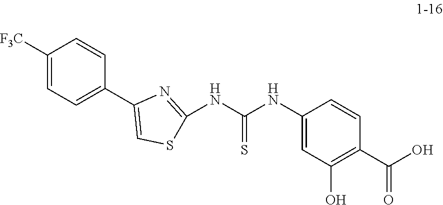 [SMILES: O=C(O)c1ccc(NC(=S)Nc2nc(-c3ccc(C(F)(F)F)cc3)cs2)cc1O]

Compound 1-16 was obtained following the procedure used to prepare compound 1-5. ¹H NMR (400 MHz, DMSO-d₆) δ 12.12 (s, 1H), δ 11.42 (s, 1H), 10.77 (s, 1H), δ 8.12 (d, J=8.0 Hz, 2H), 7.81 (dd, J=19.1, 8.4 Hz, 4H), 7.63 (d, J=2.1 Hz, 1H), 7.19 (s, 1H). LCMS (ESI) 440 [M+H]⁺, 438 [M−H]⁻. HPLC purity at 254 nm, 96.4%.

### Example 17

### Preparation of 2-hydroxy-4-(3-(4-(3-(trifluoromethyl)phenyl)thiazol-2-yl)thioureido)benzoic acid (Compound 1-17)

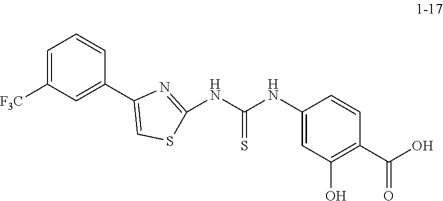 [SMILES: O=C(O)c1ccc(NC(=S)Nc2nc(-c3cccc(C(F)(F)F)c3)cs2)cc1O]

Compound 1-17 was obtained following the procedure used to prepare compound 1-5. H NMR (400 MHz, DMSO-d₆) δ 12.12 (s, 1H), δ 11.41 (s, 1H), 10.80 (s, 1H), δ 8.28-8.19 (m, 2H), 7.87-7.69 (m, 4H), 7.63 (d, J=2.1 Hz, 1H), 7.18 (s, 1H). LCMS (ESI) 440 [M+H]⁺, 438 [M−H]⁻. HPLC purity at 254 nm, 98.9%.

### Example 18

### Preparation of 2-hydroxy-4-(3-(4-(4-methoxyphenyl)thiazol-2-yl)thioureido)benzoic acid (Compound 1-18)

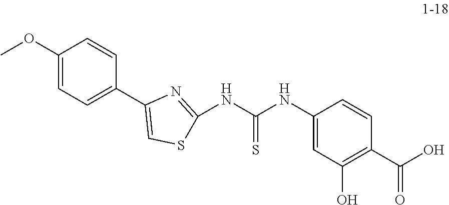 [SMILES: COc1ccc(-c2csc(NC(=S)Nc3ccc(C(=O)O)c(O)c3)n2)cc1]

Compound 1-18 was obtained following the procedure used to prepare compound 1-5. ¹H NMR (400 MHz, DMSO-d₆) δ 11.39 (s, 1H), 7.86-7.71 (m, 3H), 7.63 (s, 1H), 7.33 (s, 2H), 7.04 (dd, J=8.6, 6.6 Hz, 2H), 3.82 (s, 3H). LCMS (ESI) 402 [M+H]⁺, 400 [M−H]⁻. HPLC purity at 254 nm, 99.8%.

### Example 19

### Preparation of 2-hydroxy-4-(3-(4-(3-methoxyphenyl)thiazol-2-yl)thioureido)benzoic acid (Compound 1-19)

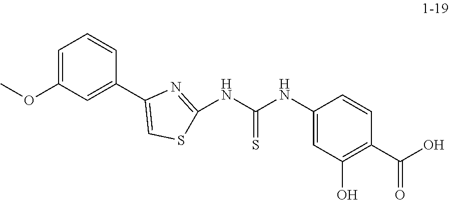 [SMILES: COc1cccc(-c2csc(NC(=S)Nc3ccc(C(=O)O)c(O)c3)n2)c1]

Compound 1-19 was obtained following the procedure used to prepare compound 1-5. ¹H NMR (400 MHz, DMSO-d₆) δ 11.40 (s, 1H), 10.81 (s, 1H), 7.76 (d, J=8.7 Hz, 1H), 7.64-7.52 (m, 2H), 7.49-7.35 (m, 3H), 7.28 (s, 1H), 6.99-6.91 (m, 1H), 3.84 (s, 3H). LCMS (ESI) 402 [M+H]⁺, 400 [M−H]⁻. HPLC purity at 254 nm, 99.8%.

### Example 20

### Preparation of 2-hydroxy-4-(3-(4-(4-hydroxyphenyl)thiazol-2-yl)thioureido)benzoic acid (Compound 1-20)

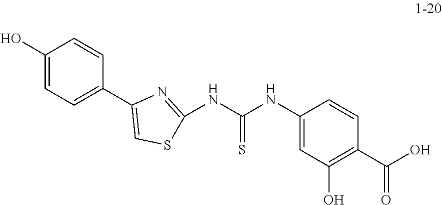 [SMILES: O=C(O)c1ccc(NC(=S)Nc2nc(-c3ccc(O)cc3)cs2)cc1O]

Compound 1-20 was obtained following the procedure used to prepare compound 1-5. ¹H NMR (400 MHz, DMSO-d₆) δ 11.38 (s, 1H), 9.73 (s, 11H), 7.80-7.56 (m, 4H), 7.50-7.12 (m, 2H), 6.85 (dd, J=8.6, 6.5 Hz, 2H). LCMS (ESI) 388 [M+H]⁺, 386 [M−H]⁻. HPLC purity at 254 nm, 99.9%.

### Example 21

### Preparation of 2-hydroxy-4-(3-(4-(3-hydroxyphenyl)thiazol-2-yl)thioureido)benzoic acid (Compound 1-21)

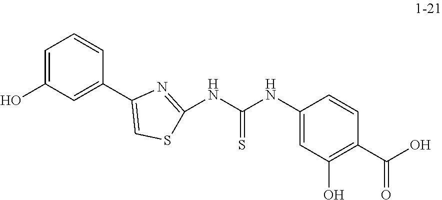 [SMILES: O=C(O)c1ccc(NC(=S)Nc2nc(-c3cccc(O)c3)cs2)cc1O]

Compound 1-21 was obtained following the procedure used to prepare compound 1-5. ¹H NMR (400 MHz, DMSO-d₆) δ 11.39 (s, 1H), 9.61 (s, 1H), 7.76 (d, J=8.7 Hz, 1H), 7.63 (d, J=2.1 Hz, 1H), 7.50-7.37 (m, 1H), 7.35-7.18 (m, 4H), 6.80 (d, J=7.7 Hz, 1H). LCMS (ESI) 388 [M+H]⁺, 386 [M−H]⁻. HPLC purity at 254 nm, 99.5%.

### Example 22

### Preparation of 4-(3-(4-(4-cyanophenyl)thiazol-2-yl)thioureido)-2-hydroxybenzoic acid (Compound 1-22)

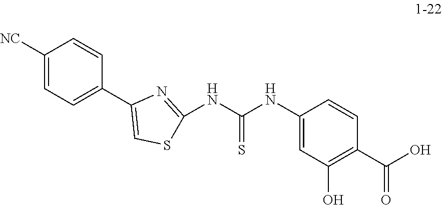 [SMILES: N#Cc1ccc(-c2csc(NC(=S)Nc3ccc(C(=O)O)c(O)c3)n2)cc1]

Compound 1-22 was obtained following the procedure used to prepare compound 1-5. ¹H NMR (400 MHz, DMSO-d₆) δ 12.07 (s, 1H), 11.36 (d, J=34.0 Hz, 1H), 10.76 (s, 1H), 8.13-8.05 (m, 2H), 7.93 (dd, J=8.2, 5.5 Hz, 2H), 7.85 (d, J=6.5 Hz, 1H), 7.79 (d, J=8.6 Hz, 1H), 7.62 (d, J=2.1 Hz, 1H), 7.18 (s, 1H). LCMS (ESI) 397 [M+H]⁺, 395 [M−H]⁻. HPLC purity at 254 nm, 98.5%.

### Example 23

### Preparation of 2-hydroxy-4-(3-(4-(4-nitrophenyl)thiazol-2-yl)thioureido)benzoic acid (Compound 1-23)

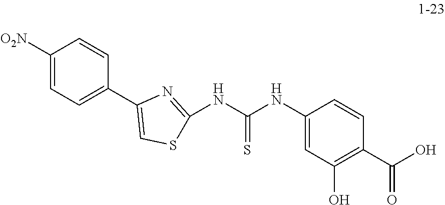 [SMILES: O=C(O)c1ccc(NC(=S)Nc2nc(-c3ccc([N+](=O)[O-])cc3)cs2)cc1O]

Compound 1-23 was obtained following the procedure used to prepare compound 1-5. ¹H NMR (400 MHz, DMSO-d₆) δ 12.12 (s, 1H), 11.41 (s, 1H), 10.75 (s, 1H), 8.39-8.30 (m, 2H), 8.21-8.15 (m, 2H), 7.95 (s, 1H), 7.80 (d, J=8.6 Hz, 1H), 7.62 (d, J=2.1 Hz, 1H), 7.17 (s, 1H). LCMS (ESI) 417 [M+H]⁺, 415 [M−H]⁻. HPLC purity at 254 nm, 99.2%.

### Example 24

### Preparation of 4-(3-(4-(3-acetamidophenyl)thiazol-2-yl)thioureido)-2-hydroxybenzoic acid (Compound 1-24)

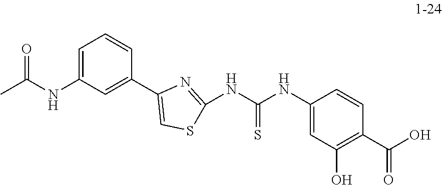 [SMILES: CC(=O)Nc1cccc(-c2csc(NC(=S)Nc3ccc(C(=O)O)c(O)c3)n2)c1]

Compound 1-24 was obtained following the procedure used to prepare compound 1-5. ¹H NMR (400 MHz, DMSO-d₆) δ 10.05 (s, 1H), 8.26-7.10 (m, 8H), 2.08 (s, 3H). LCMS (ESI) 429 [M+H]f, 427 [M−H]⁻. HPLC purity at 254 nm, 99.9%.

### Example 25

### Preparation of 4-(3-(4-(2-acetamidophenyl)thiazol-2-yl)thioureido)-2-hydroxybenzoic acid (Compound 1-25)

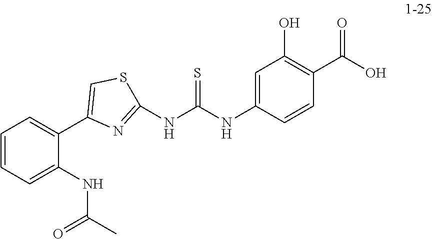 [SMILES: CC(=O)Nc1ccccc1-c1csc(NC(=S)Nc2ccc(C(=O)O)c(O)c2)n1]

Compound 1-25 was obtained following the procedure used to prepare compound 1-5. ¹H NMR (400 MHz, DMSO-d₆) δ 13.57 (s, 1H), 12.07 (s, 1H), 11.35 (s, 1H), 8.29-6.87 (m, 8H), 2.16-2.02 (m, 3H). LCMS (ESI) 429 [M+H]⁺, 427 [M−H]⁻. HPLC purity at 254 nm, 98.2%.

### Example 26

### Preparation of 4-(3-(4-(4-bromophenyl)thiazol-2-yl)thioureido)-2-hydroxybenzoic acid (Compound 1-26)

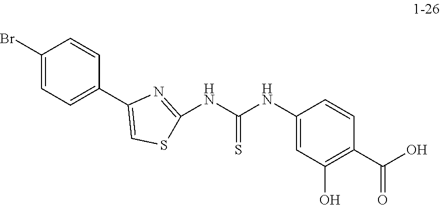 [SMILES: O=C(O)c1ccc(NC(=S)Nc2nc(-c3ccc(Br)cc3)cs2)cc1O]

Compound 1-26 was obtained following the procedure used to prepare compound 1-5. ¹H NMR (400 MHz, DMSO-d₆) δ 12.05 (s, 1H), 11.40 (s, 1H), 10.81 (s, 1H), 7.89-7.83 (m, 2H), 7.77 (d, J=8.7 Hz, 1H), 7.72-7.54 (m, 4H), 7.21 (s, 1H). LCMS (ESI) 450 [M+H]⁺. HPLC purity at 254 nm, 99.9%.

### Example 27

### Preparation of 4-(3-(4-(4-chlorophenyl)thiazol-2-yl)thioureido)-2-hydroxybenzoic acid (Compound 1-27)

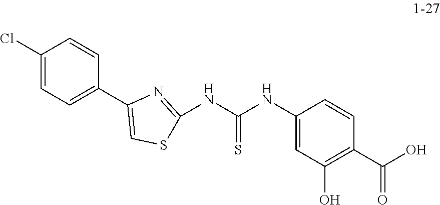 [SMILES: O=C(O)c1ccc(NC(=S)Nc2nc(-c3ccc(Cl)cc3)cs2)cc1O]

Compound 1-27 was obtained following the procedure used to prepare compound 1-5. ¹H NMR (400 MHz, DMSO-d₆) δ 12.03 (s, 1H), δ 11.40 (s, 1H), 10.81 (s, 1H), 7.98-7.88 (m, 2H), 7.77 (d, J=8.7 Hz, 1H), 7.69-7.49 (m, 4H), 7.21 (s, 1H). LCMS (ESI) 406 [M+H]⁺, 404 [M−H]⁻. HPLC purity at 254 nm, 99.0%.

### Example 28

### Preparation of 2-hydroxy-4-(3-(4-(4-(N-methylsulfamoyl)phenyl)thiazol-2-yl)thioureido)benzoic acid (Compound 1-28)

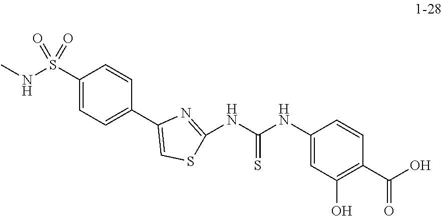 [SMILES: CNS(=O)(=O)c1ccc(-c2csc(NC(=S)Nc3ccc(C(=O)O)c(O)c3)n2)cc1]

Compound 1-28 was obtained following the procedure used to prepare compound 1-5. ¹H NMR (400 MHz, DMSO-d₆) δ 12.06 (s, 1H), 11.41 (s, 1H), 10.79 (s, 1H), 8.11 (d, J=8.2 Hz, 2H), 7.92-7.72 (m, 4H), 7.63 (d, J=2.1 Hz, 1H), 7.54-7.05 (m, 2H), 2.45 (dd, J=5.0, 2.6 Hz, 3H). LCMS (ESI) 465 [M+H]⁺, 463 [M−H]⁻. HPLC purity at 254 nm, 98.9%.

### Example 29

### Preparation of 2-hydroxy-4-(3-(4-(3-(N-methylsulfamoyl)phenyl)thiazol-2-yl)thioureido)benzoic acid (Compound 1-29)

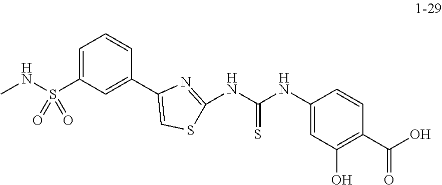 [SMILES: CNS(=O)(=O)c1cccc(-c2csc(NC(=S)Nc3ccc(C(=O)O)c(O)c3)n2)c1]

Compound 1-29 was obtained following the procedure used to prepare compound 1-5. ¹H NMR (400 MHz, DMSO-d₆) δ 12.06 (s, 1H), 11.41 (s, 1H), 10.72 (s, 1H), 8.34 (s, 1H), 8.16 (d, J=7.6 Hz, 1H), 7.87-7.66 (m, 5H), 7.49 (dd, J=7.4, 3.4 Hz, 1H), 7.16 (d, J=20.1 Hz, 1H), 2.46 (dd, J=5.0, 1.7 Hz, 3H). LCMS (ESI) 465 [M+H]⁺, 463 [M−H]⁻. HPLC purity at 254 nm, 98.8%.

### Example 30

### Preparation of 4-(3-(4-(3,4-difluorophenyl)thiazol-2-yl)thioureido)-2-hydroxybenzoic acid (Compound 1-30)

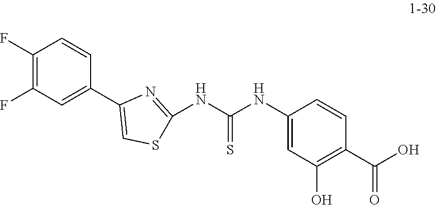 [SMILES: O=C(O)c1ccc(NC(=S)Nc2nc(-c3ccc(F)c(F)c3)cs2)cc1O]

Compound 1-30 was obtained following the procedure used to prepare compound 1-5. ¹H NMR (400 MHz, DMSO-d₆) δ 12.03 (s, 1H), 11.40 (s, 1H), 10.79 (s, 1H), 7.94 (t, J=9.9 Hz, 1H), 7.84-7.40 (m, 5H), 7.18 (s, 1H). LCMS (ESI) 408 [M+H]⁺, 406 [M−H]⁻. HPLC purity at 254 nm, 99.5%.

### Example 31

### Preparation of 4-(3-(4-(2,4-difluorophenyl)thiazol-2-yl)thioureido)-2-hydroxybenzoic acid (Compound 1-31)

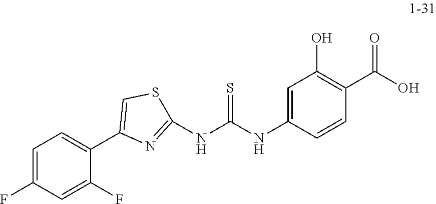 [SMILES: O=C(O)c1ccc(NC(=S)Nc2nc(-c3ccc(F)cc3F)cs2)cc1O]

Compound 1-31 was obtained following the procedure used to prepare compound 1-5. ¹H NMR (400 MHz, DMSO-d₆) δ 11.39 (s, 1H), 8.02 (s, 1H), 7.75 (dd, J=19.7, 8.5 Hz, 1H), 7.62 (d, J=2.1 Hz, 1H), 7.41 (d, J=13.1 Hz, 2H), 7.20 (d, J=31.9 Hz, 2H). LCMS (ESI) 408 [M+H]⁺, 406 [M−H]⁻. HPLC purity at 254 nm, 99.0%.

### Example 32

### Preparation of 4-(3-(4-(4-cyano-3-fluorophenyl)thiazol-2-yl)thioureido)-2-hydroxybenzoic acid (Compound 1-32)

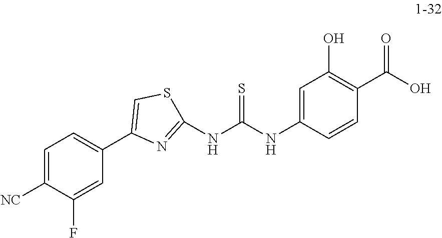 [SMILES: N#Cc1ccc(-c2csc(NC(=S)Nc3ccc(C(=O)O)c(O)c3)n2)cc1F]

Compound 1-32 was obtained following the procedure used to prepare compound 1-5. ¹H NMR (400 MHz, DMSO-d₆) δ 12.14 (s, 1H), 11.39 (s, 1H), 10.73 (s, 1H), 8.01-7.93 (m, 4H), 7.76 (dd, J=18.3, 8.6 Hz, 1H), 7.59 (d, J=2.1 Hz, 1H), 7.20-7.13 (m, 1H). LCMS (ESI) 415 [M+H]⁴, 413 [M−H]⁻. HPLC purity at 254 nm, 98.8%.

### Example 33

### Preparation of 4-(3-(4-(4-cyano-2-fluorophenyl)thiazol-2-yl)thioureido)-2-hydroxybenzoic acid (Compound 1-33)

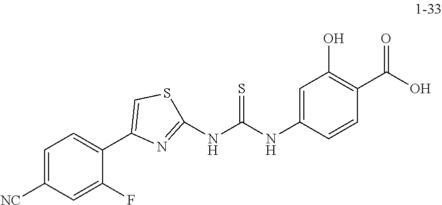 [SMILES: N#Cc1ccc(-c2csc(NC(=S)Nc3ccc(C(=O)O)c(O)c3)n2)c(F)c1]

Compound 1-33 was obtained following the procedure used to prepare compound 1-5. ¹H NMR (400 MHz, DMSO-d₆) δ 12.08 (s, 1H), 11.40 (s, 1H), 10.77 (s, 1H), 8.19 (q, J=7.8 Hz, 1H), 8.05-7.94 (m, 1H), 7.87-7.68 (m, 3H), 7.60 (d, J=2.1 Hz, 1H), 7.15 (d, J=9.2 Hz, 1H). LCMS (ESI) 415 [M+H]⁺, 413 [M−H]⁻. HPLC purity at 254 nm, 99.9%.

### Example 34

### Preparation of 4-(3-(4-(2-fluoro-4-nitrophenyl)thiazol-2-yl)thioureido)-2-hydroxybenzoic acid (Compound 1-34)

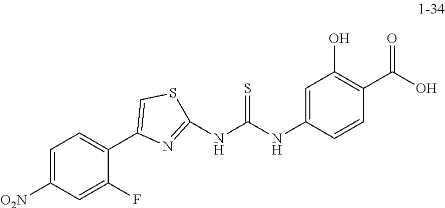 [SMILES: O=C(O)c1ccc(NC(=S)Nc2nc(-c3ccc([N+](=O)[O-])cc3F)cs2)cc1O]

Compound 1-34 was obtained following the procedure used to prepare compound 1-5. ¹H NMR (400 MHz, DMSO-d₆) δ 12.15 (s, 1H), 11.41 (s, OH), 10.74 (s, 1H), 8.40-8.00 (m, 4H), 7.85-7.75 (m, 1H), 7.60 (d, J=2.1 Hz, 1H), 7.15 (d, J=9.0 Hz, 1H). LCMS (ESI) 435 [M+H]⁺, 433 [M−H]⁻. HPLC purity at 254 nm, 99.8%.

### Example 35

### Preparation of 4-(3-(4-(3-fluoro-4-nitrophenyl)thiazol-2-yl)thioureido)-2-hydroxybenzoic acid (Compound 1-35)

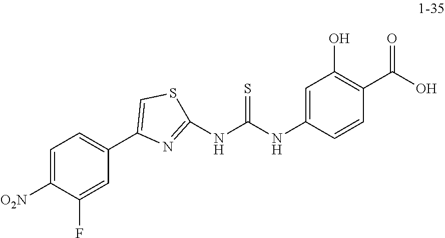 [SMILES: O=C(O)c1ccc(NC(=S)Nc2nc(-c3ccc([N+](=O)[O-])c(F)c3)cs2)cc1O]

Compound 1-35 was obtained following the procedure used to prepare compound 1-5. ¹H NMR (400 MHz, DMSO-d₆) δ 12.09 (s, 1H), 11.40 (s, 1H), 10.73 (s, 1H), 8.33-8.22 (m, 1H), 8.14-7.91 (m, 3H), 7.77 (dd, J=18.4, 8.5 Hz, 1H), 7.60 (d, J=2.1 Hz, 1H), 7.25-7.03 (m, 1H). LCMS (ESI) 435 [M+H]⁺, 433 [M−H]⁻. HPLC purity at 254 nm, 99.9%.

### Example 36

### Preparation of 4-(3-(4-(4-fluoro-3-nitrophenyl)thiazol-2-yl)thioureido)-2-hydroxybenzoic acid (Compound 1-36)

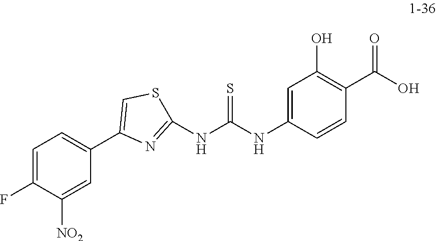 [SMILES: O=C(O)c1ccc(NC(=S)Nc2nc(-c3ccc(F)c([N+](=O)[O-])c3)cs2)cc1O]

Compound 1-36 was obtained following the procedure used to prepare compound 1-5. ¹H NMR (400 MHz, DMSO-d₆) δ 12.10 (s, 1H), 11.41 (s, 1H), 10.75 (s, 1H), 8.64 (dt, J=7.3, 2.3 Hz, 1H), 8.32 (ddt, J=8.8, 4.5, 2.3 Hz, 1H), 7.89-7.58 (m, 4H), 7.12 (s, 1H). LCMS (ESI) 435 [M+H]⁺, 433 [M−H]⁻. HPLC purity at 254 nm, 97.2%.

### Example 37

### Preparation of 4-(3-(4-(3-(N-cyclopentylsulfamoyl)-4-fluorophenyl)thiazol-2-yl)thioureido)-2-hydroxybenzoic acid (Compound 1-37)

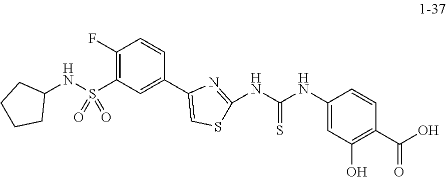 [SMILES: O=C(O)c1ccc(NC(=S)Nc2nc(-c3ccc(F)c(S(=O)(=O)NC4CCCC4)c3)cs2)cc1O]

Compound 1-37 was obtained following the procedure used to prepare compound 1-5. ¹H NMR (400 MHz, DMSO-d₆) δ 12.13 (s, 1H), 11.43 (s, 1H), 10.78 (s, 1H), 8.36 (d, J=6.8 Hz, 1H), 8.19 (ddd, J=8.8, 4.6, 2.4 Hz, 1H), 8.03 (t, J=7.6 Hz, 1H), 7.82-7.65 (m, 3H), 7.61-7.46 (m, 1H), 7.14 (s, 1H), 3.55 (q, J=7.3 Hz, 1H), 1.69-1.49 (m, 4H), 1.45-1.30 (m, 4H). LCMS (ESI) 537 [M+H]⁺, 535 [M−H]⁻. HPLC purity at 254 nm, 99.9%.

### Example 38

### Preparation of 4-(3-(4-(3-(N-benzylsulfamoyl)-4-fluorophenyl)thiazol-2-yl)thioureido)-2-hydroxybenzoic acid (Compound 1-38)

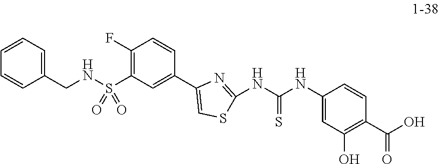 [SMILES: O=C(O)c1ccc(NC(=S)Nc2nc(-c3ccc(F)c(S(=O)(=O)NCc4ccccc4)c3)cs2)cc1O]

Compound 1-38 was obtained following the procedure used to prepare compound 1-5. ¹H NMR (400 MHz, DMSO-d₆) δ 12.12 (s, 1H), 11.42 (s, 1H), 10.80 (s, 1H), 8.57 (q, J=6.2 Hz, 1H), 8.28 (s, 1H), 8.13 (s, 1H), 7.82-7.64 (m, 3H), 7.51-7.41 (m, 1H), 7.27-7.03 (m, 6H), 4.15 (d, J=6.2 Hz, 2H). LCMS (ESI) 559 [M+H]⁺, 557 [M−H]⁻. HPLC purity at 254 nm, 99.9%.

### Example 39

### Preparation of 4-(3-(4-(3-(N-benzylsulfamoyl)-4-nitrophenyl)thiazol-2-yl)thioureido)-2-hydroxybenzoic acid (Compound 1-39)

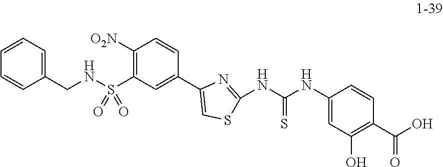 [SMILES: O=C(O)c1ccc(NC(=S)Nc2nc(-c3ccc([N+](=O)[O-])c(S(=O)(=O)NCc4ccccc4)c3)cs2)cc1O]

Compound 1-39 was obtained following the procedure used to prepare compound 1-5. H NMR (400 MHz, DMSO-d₆) δ 12.17 (s, 1H), 11.41 (s, 1H), 10.77 (s, 1H), 8.57 (t, J=6.2 Hz, 1H), 8.36 (d, J=1.9 Hz, 1H), 8.24 (dd, J=8.3, 2.0 Hz, 1H), 8.03 (dd, J=8.3, 4.3 Hz, 1H), 7.88 (s, 1H), 7.81-7.63 (m, 2H), 7.28-7.06 (m, 6H), 4.24 (d, J=6.2 Hz, 2H). LCMS (ESI) 586 [M+H]⁺, 584 [M−H]⁻. HPLC purity at 254 nm, 97.7%.

### Example 40

### Preparation of ethyl 2-hydroxy-4-(3-(4-(4-nitrophenyl)thiazol-2-yl)thioureido)benzoate (Compound 1-40)

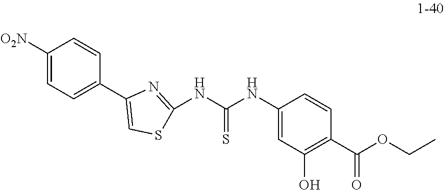 [SMILES: CCOC(=O)c1ccc(NC(=S)Nc2nc(-c3ccc([N+](=O)[O-])cc3)cs2)cc1O]

Compound 1-40 was obtained following the procedure used to prepare compound 1-6. ¹H NMR (400 MHz, DMSO-d₆) δ 10.74 (s, 1H), 8.38-8.28 (m, 2H), 8.21-8.11 (m, 2H), 7.92 (s, 1H), 7.79 (d, J=8.7 Hz, 1H), 7.68 (s, 1H), 7.20 (s, 1H), 4.37 (q, J=7.1 Hz, 2H), 1.35 (t, J=7.1 Hz, 3H). LCMS (ESI) 445 [M+H]⁺, 443 [M−H]⁻. HPLC purity at 254 nm, 99.9%.

### Example 41

### Preparation of ethyl 4-(3-(4-(4-cyanophenyl)thiazol-2-yl)thioureido)-2-hydroxybenzoate (Compound 1-41)

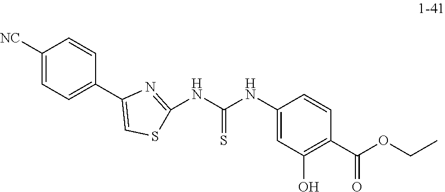 [SMILES: CCOC(=O)c1ccc(NC(=S)Nc2nc(-c3ccc(C#N)cc3)cs2)cc1O]

Compound 1-41 was obtained following the procedure used to prepare compound 1-6. ¹H NMR (400 MHz, DMSO-d₆) δ 12.15 (s, 1H), 11.01 (s, 1H), 10.74 (s, 1H), 8.13-8.05 (m, 2H), 8.00-7.73 (m, 4H), 7.68 (s, 1H), 7.20 (s, 1H), 4.37 (q, J=7.0 Hz, 2H), 1.35 (t, J=7.1 Hz, 3H). LCMS (ESI) 425 [M+H]⁺, 423 [M−H]⁻. HPLC purity at 254 nm, 99.6%.

### Example 42

### Preparation of ethyl 4-(3-(4-(3-(N-benzylsulfamoyl)-4-nitrophenyl)thiazol-2-yl)thioureido)-2-hydroxybenzoate (compound 1-42)

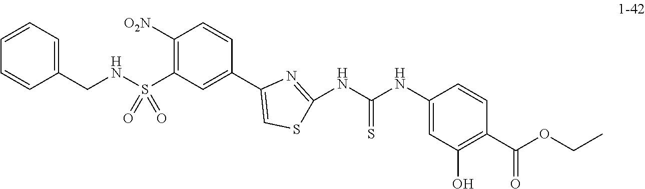 [SMILES: CCOC(=O)c1ccc(NC(=S)Nc2nc(-c3ccc([N+](=O)[O-])c(S(=O)(=O)NCc4ccccc4)c3)cs2)cc1O]

Compound 1-42 was obtained following the procedure used to prepare compound 1-6. ¹H NMR (400 MHz, DMSO-d₆) δ 12.16 (s, 1H), 10.74 (s, 1H), 8.57 (t, J=6.2 Hz, 1H), 8.36 (s, 1H), 8.25 (dd, J=8.4, 1.9 Hz, 1H), 8.03 (d, J=8.4 Hz, 1H), 7.90 (s, 1H), 7.79 (d, J=8.7 Hz, 1H), 7.70 (s, 1H), 7.28-7.08 (m, 6H), 4.37 (q, J=7.1 Hz, 2H), 4.24 (d, J=6.1 Hz, 2H), 1.35 (t, J=7.1 Hz, 3H). LCMS (ESI) 614 [M+H]⁺, 612 [M−H]⁻. HPLC purity at 254 nm, 99.7%.

### Example 43

### Preparation of ethyl4-(3-(4-(3-(N-benzylsulfamoyl)-4-fluorophenyl)thiazol-2-yl)thioureido)-2-hydroxybenzoate (Compound 1-43)

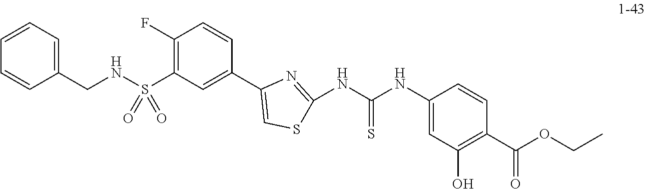 [SMILES: CCOC(=O)c1ccc(NC(=S)Nc2nc(-c3ccc(F)c(S(=O)(=O)NCc4ccccc4)c3)cs2)cc1O]

Compound 1-43 was obtained following the procedure used to prepare compound 1-6. ¹H NMR (400 MHz, DMSO-d₆) δ 12.20 (s, 1H), 10.96 (s, 1H), 10.75 (s, 1H), 8.56 (t, J=6.3 Hz, 1H), 8.28 (s, 1H), 8.13 (ddd, J=7.9, 4.3, 2.2 Hz, 1H), 7.86-7.65 (m, 3H), 7.46 (t, J=9.3 Hz, 1H), 7.30-7.04 (m, 6H), 4.37 (q, J=7.1 Hz, 2H), 4.16 (d, J=6.2 Hz, 2H), 1.35 (t, J=7.1 Hz, 3H). LCMS (ESI) 587 [M+H]⁺, 585 [M−H]⁻. HPLC purity at 254 nm, 99.9%.

### Example 44

### Preparation of (3-((4-(2-((4-(ethoxycarbonyl)-3-hydroxyphenyl)amino)thiazol-4-yl)phenyl)amino)-3-oxopropyl)triphenylphosphonium (Compound 1-44)

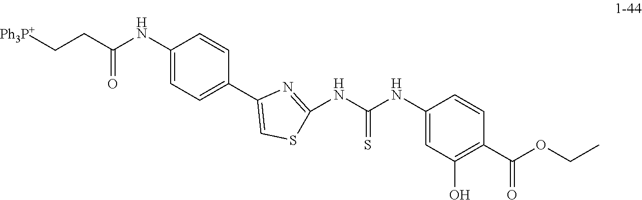

Compound 1-44 was obtained following the procedure used to prepare compound 1-6. ¹H NMR (400 MHz, DMSO-d6) δ 10.87 (s, 1H), 10.71 (s, 1H), 10.19 (s, 1H), 7.98-7.73 (m, 18H), 7.64-7.52 (m, 3H), 7.37 (s, 1H), 7.14 (dd, J=8.8, 2.2 Hz, 1H), 4.35 (q, J=7.1 Hz, 2H), 4.00-3.83 (m, 2H), 2.84-2.71 (m, 2H), 1.35 (t, J=7.1 Hz, 3H). LCMS (ESI) 673 [M+H]⁺, 671 [M−H]⁻. HPLC purity at 254 nm, 99.8%.

### INCORPORATION BY REFERENCE

The entire disclosure of each of the patent documents and scientific articles referred to herein is incorporated by reference for all purposes. The following references are herein incorporated by reference in their entireties:
Dankort, D., Curley, D. P., Cartlidge, R. A., Nelson, B., Karnezis, A. N., Damsky, W. E., Jr., You, M. J., DePinho, R. A., McMahon, M., and Bosenberg, M. (2009). Braf(V600E) cooperates with Pten loss to induce metastatic melanoma. Nat Genet 41, 544-552.
Du, J., Zhou, Y., Su, X., Yu, J. J., Khan, S., Jiang, H., Kim, J., Woo, J., Kim, J. H., Choi, B. H., et al. (2011). SIRT5 is a NAD-dependent protein lysine demalonylase and desuccinylase. Science 334, 806-809.
Gaspar, N., Hawkins, D. S., Dirksen, U., Lewis, I. J., Ferrari, S., Le Deley, M. C., Kovar, H., Grimer, R., Whelan, J., Claude, L., et al. (2015). Ewing Sarcoma: Current Management and Future Approaches Through Collaboration. J Clin Oncol 33, 3036-3046.
Giblin, W., Skinner, M. E., and Lombard, D. B. (2014). Sirtuins: guardians of mammalian healthspan. Trends Genet 30, 271-286.
Guetschow, E. D., Kumar, S., Lombard, D. B., and Kennedy, R. T. (2016). Identification of sirtuin 5 inhibitors by ultrafast microchip electrophoresis using nanoliter volume samples. Analytical and bioanalytical chemistry 408, 721-731.
Hodis, E., Watson, I. R., Kryukov, G. V., Arold, S. T., Imielinski, M., Theurillat, J. P., Nickerson, E., Auclair, D., Li, L., Place, C., et al. (2012). A landscape of driver mutations in melanoma. Cell 150, 251-263.
Hudson, M. M., Ness, K. K., Gurney, J. G., Mulrooney, D. A., Chemaitilly, W., Krull, K. R., Green, D. M., Armstrong, G. T., Nottage, K. A., Jones, K. E., et al. (2013). Clinical ascertainment of health outcomes among adults treated for childhood cancer. JAMA 309, 2371-2381.
Li, F., He, X., Ye, D., Lin, Y., Yu, H., Yao, C., Huang, L., Zhang, J., Wang, F., Xu, S., et al. (2015). NADP(+)-IDH Mutations Promote Hypersuccinylation that Impairs Mitochondria Respiration and Induces Apoptosis Resistance. Mol Cell 60, 661-675.
Lin, Z. F., Xu, H. B., Wang, J. Y., Lin, Q., Ruan, Z., Liu, F. B., Jin, W., Huang, H. H., and Chen, X. (2013). SIRT5 desuccinylates and activates SOD1 to eliminate ROS. Biochem Biophys Res Commun 441, 191-195.
Lombard, D. B., Alt, F. W., Cheng, H. L., Bunkenborg, J., Streeper, R. S., Mostoslavsky, R., Kim, J., Yancopoulos, G., Valenzuela, D., Murphy, A., et al. (2007). Mammalian Sir2 homolog SIRT3 regulates global mitochondrial lysine acetylation. Mol Cell Biol 27, 8807-8814.
Lombard, D. B., Tishkoff, D. X., and Zwaans, B. M. (2012). Mitochondrial regulation by protein acetylation. In Mitochondrial Signaling in Health and Disease, E. Cadenas, S. Orrenius, and L. Packer, eds. (London: Taylor and Francis), pp. 269-298.
Lu, W., Zuo, Y., Feng, Y., and Zhang, M. (2014). SIRT5 facilitates cancer cell growth and drug resistance in non-small cell lung cancer. Tumour biology: the journal of the International Society for Oncodevelopmental Biology and Medicine.
Nakagawa, T., Lomb, D. J., Haigis, M. C., and Guarente, L. (2009). SIRT5 Deacetylates carbamoyl phosphate synthetase 1 and regulates the urea cycle. Cell 137, 560-570.
Nishida, Y., Rardin, M. J., Carrico, C., He, W., Sahu, A. K., Gut, P., Najjar, R., Fitch, M., Hellerstein, M., Gibson, B. W., et al. (2015). SIRT5 Regulates both Cytosolic and Mitochondrial Protein Malonylation with Glycolysis as a Major Target. Mol Cell 59, 321-332.
Park, J., Chen, Y., Tishkoff, D. X., Peng, C., Tan, M., Dai, L., Xie, Z., Zhang, Y., Zwaans, B. M., Skinner, M. E., et al. (2013). SIRT5-mediated lysine desuccinylation impacts diverse metabolic pathways. Mol Cell 50, 919-930.
Peng, C., Lu, Z., Xie, Z., Cheng, Z., Chen, Y., Tan, M., Luo, H., Zhang, Y., He, W., Yang, K., et al. (2011). The first identification of lysine malonylation substrates and its regulatory enzyme. Mol Cell Proteomics 10, M111 012658.
Polletta, L., Vernucci, E., Carnevale, I., Arcangeli, T., Rotili, D., Palmerio, S., Steegborn, C., Nowak, T., Schutkowski, M., Pellegrini, L., et al. (2015). SIRT5 regulation of ammonia-induced autophagy and mitophagy. Autophagy 11, 253-270.
Rardin, M. J., He, W., Nishida, Y., Newman, J. C., Carrico, C., Danielson, S. R., Guo, A., Gut, P., Sahu, A. K., Li, B., et al. (2013). SIRT5 Regulates the Mitochondrial Lysine Succinylome and Metabolic Networks. Cell Metab 18, 920-933.
Roessler, C., Nowak, T., Pannek, M., Gertz, M., Nguyen, G. T., Scharfe, M., Born, I., Sippl, W., Steegborn, C., and Schutkowski, M. (2014). Chemical Probing of the Human Sirtuin 5 Active Site Reveals Its Substrate Acyl Specificity and Peptide-Based Inhibitors. Angew Chem Int Ed Engl.
Roth, M., and Chen, W. Y. (2014). Sorting out functions of sirtuins in cancer. Oncogene 33, 1609-1620.
Sadhukhan, S., Liu, X., Ryu, D., Nelson, O. D., Stupinski, J. A., Li, Z., Chen, W., Zhang, S., Weiss, R. S., Locasale, J. W., et al. (2016). Metabolomics-assisted proteomics identifies succinylation and SIRT5 as important regulators of cardiac function. Proc Natl Acad Sci USA 113, 4320-4325.
Tan, M., Peng, C., Anderson, K. A., Chhoy, P., Xie, Z., Dai, L., Park, J., Chen, Y., Huang, H., Zhang, Y., et al. (2014). Lysine glutarylation is a protein posttranslational modification regulated by SIRT5. Cell Metab 19, 605-617.
Weinert, B. T., Scholz, C., Wagner, S. A., Iesmantavicius, V., Su, D., Daniel, J. A., and Choudhary, C. (2013). Lysine succinylation is a frequently occurring modification in prokaryotes and eukaryotes and extensively overlaps with acetylation. Cell Rep 4, 842-851.
Xu, Y. S., Liang, J. J., Wang, Y., Zhao, X. J., Xu, L., Xu, Y. Y., Zou, Q. C., Zhang, J. M., Tu, C. E., Cui, Y. G., et al. (2016). STAT3 Undergoes Acetylation-dependent Mitochondrial Translocation to Regulate Pyruvate Metabolism. Sci Rep 6, 39517.
Yang, L., He, Y., Chen, Q., Qian, S., and Wang, Z. (2016). Design and Synthesis of New 9-Substituted Norharmane Derivatives as Potential SIRT5 Inhibitors. J Heterocyclic Chem 54,
Yu, J., Sadhukhan, S., Noriega, L. G., Moullan, N., He, B., Weiss, R. S., Lin, H., Schoonjans, K., and Auwerx, J. (2013). Metabolic characterization of a SIRT5 deficient mouse model. Sci Rep 3, 2806.
Zhou, L., Wang, F., Sun, R., Chen, X., Zhang, M., Xu, Q., Wang, Y., Wang, S., Xiong, Y., Guan, K. L., et al. (2016). SIRT5 promotes IDH2 desuccinylation and G6PD deglutarylation to enhance cellular antioxidant defense. EMBO Rep 17, 811-822.
Zwaans, B. M., and Lombard, D. B. (2014). Interplay between sirtuins, MYC and hypoxia-inducible factor in cancer-associated metabolic reprogramming. Dis Model Mech 7, 1023-1032.
Kumar, S., and Lombard, D. B. (2018). Functions of the sirtuin deacylase SIRT5 in normal physiology and pathobiology. Crit Rev Biochem Mol Biol 53, 311-334.
Yang, X., Wang, Z., Li, X., Liu, B., Liu, M., Liu, L., Chen, S., Ren, M., Wang, Y., Yu, M., Wang, B., Zou, J., Zhu, W. G., Yin, Y., Gu, W., and Luo, J. (2018). SHMT2 desuccinylation by SIRT5 drives cancer cell proliferation. Cancer Res 78, 372-386.
Xiangyun, Y., Xiaomin, N., Linping, G., Yunhua, X., Ziming, L., Yongfeng, Y., Zhiwei, C., and Shun, L. (2017). Desuccinylation of pyruvate kinase M2 by SIRT5 contributes to antioxidant response and tumor growth. Oncotarget 8, 6984-6993.
Wang, Y. Q., Wang, H. L., Xu, J., Tan, J., Fu, L. N., Wang, J. L., Zou, T. H., Sun, D. F., Gao, Q. Y., Chen, Y. X., and Fang, J. Y. (2018). Sirtuin5 contributes to colorectal carcinogenesis by enhancing glutaminolysis in a deglutarylation-dependent manner. Nat Commun 9, 545.
Ren, M., Yang, X., Bie, J., Wang, Z., Liu, M., Li, Y., Shao, G., and Luo, J. (2020). Citrate synthase desuccinylation by SIRT5 promotes colon cancer cell proliferation and migration. Biol Chem 401, 1031-1039.
Greene, K. S., Lukey, M. J., Wang, X., Blank, B., Druso, J. E., Lin, M. J., Stalnecker, C. A., Zhang, C., Negron Abril, Y., Erickson, J. W., Wilson, K. F., Lin, H., Weiss, R. S., and Cerione, R. A. (2019). SIRT5 stabilizes mitochondrial glutaminase and supports breast cancer tumorigenesis. Proc Natl Acad Sci USA.
Giblin, W., Bringman-Rodenbarger, L., Guo, A. H., Kumar, S., Monovich, A. C., Mostafa, A. M., Skinner, M. E., Azar, M., Mady, A. S., Chung, C. H., Kadambi, N., Melong, K. A., Lee, H. J., Zhang, L., Sajjakulnukit, P., Trefely, S., Varner, E. L., Iyer, S., Wang, M., Wilmott, J. S., Soyer, H. P., Sturm, R. A., Pritchard, A. L., Andea, A. A., Scolyer, R. A., Stark, M. S., Scott, D. A., Fullen, D. R., Bosenberg, M. W., Chandrasekaran, S., Nikolovska-Coleska, Z., Verhaegen, M. E., Snyder, N. W., Rivera, M. N., Osterman, A. L., Lyssiotis, C. A., and Lombard, D. B. (2021). The deacylase SIRT5 supports melanoma viability by influencing chromatin dynamics. J Clin Invest 131.
Abril, Y. L. N., Fernandez, I. R., Hong, J. Y., Chiang, Y. L., Kutateladze, D. A., Zhao, Q., Yang, M., Hu, J., Sadhukhan, S., Li, B., He, B., Remick, B., Bai, J. J., Mullmann, J., Wang, F., Maymi, V., Dhawan, R., Auwerx, J., Southard, T., Cerione, R. A., Lin, H., and Weiss, R. S. (2021). Pharmacological and genetic perturbation establish SIRT5 as a promising target in breast cancer. Oncogene 40, 1644-1658.
Yan, D., Franzini, A., Pomicter, A. D., Halverson, B. J., Antelope, O., Mason, C. C., Ahmann, J. M., Senina, A. V., Vellore, N. A., Jones, C. L., Zabriskie, M. S., Than, H., Xiao, M. J., van Scoyk, A., Patel, A. B., Clair, P. M., Heaton, W. L., Owen, S. C., Andersen, J. L., Egbert, C. M., Reisz, J. A., D'Alessandro, A., Cox, J. E., Gantz, K. C., Redwine, H. M., Iyer, S. M., Khorashad, J. S., Rajabi, N., Olsen, C. A., O'Hare, T., and Deininger, M. W. (2021). Sirt5 is a druggable metabolic vulnerability in acute myeloid leukemia. Blood Cancer Discov 2, 266-287.
Chen, X. F., Tian, M. X., Sun, R. Q., Zhang, M. L., Zhou, L. S., Jin, L., Chen, L. L., Zhou, W. J., Duan, K. L., Chen, Y. J., Gao, C., Cheng, Z. L., Wang, F., Zhang, J. Y., Sun, Y. P., Yu, H. X., Zhao, Y. Z., Yang, Y., Liu, W. R., Shi, Y. H., Xiong, Y., Guan, K. L., and Ye, D. (2018). SIRT5 inhibits peroxisomal ACOX1 to prevent oxidative damage and is downregulated in liver cancer. EMBO Rep 19.
Li, F., He, X., Ye, D., Lin, Y., Yu, H., Yao, C., Huang, L., Zhang, J., Wang, F., Xu, S., Wu, X., Liu, L., Yang, C., Shi, J., He, X., Liu, J., Qu, Y., Guo, F., Zhao, J., Xu, W., and Zhao, S. (2015). NADP(+)-IDH Mutations Promote Hypersuccinylation that impairs mitochondria respiration and induces apoptosis resistance. Mol Cell 60, 661-675.
Hu, T., Shukla, S. K., Vernucci, E., Hea, C., Wang, D., King, R. J., Jha, K., Siddhanta, K., Mullen, N. J., Attri, K. S., . . . , and Pankaj K. Singha (2021). Metabolic rewiring by loss of Sirt5 promotes Kras-induced pancreatic cancer progression. Gastroenterology.
Toure, M.; Crews, C. M. (2016). Small-molecule PROTACS: New approaches to protein degradation. Angew. Chem., Int. Ed. 55, 1966-1973.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound described by Formula (1), Formula (2), or Formula (3) are provided: 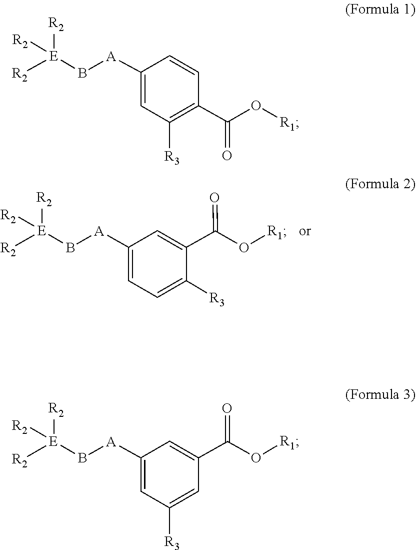
including pharmaceutically acceptable salts, solvates, PROTACs, and/or prodrugs thereof; wherein for R1, R2, R3, A, B and E independently include any chemical moiety that permits the resulting compound capable of inhibiting and/or degrading sirtuin activity.

2. The compound of claim 1, wherein the sirtuin activity is one or more of SIRT1 activity, SIRT2 activity, SIRT3 activity, SIRT4 activity, SIRT5 activity, SIRT6 activity, and SIRT7 activity.

3. The compound of claim 1, wherein R1, R2, R3, A, B and E independently include any chemical moiety that permits the resulting compound capable of one or more of:
inhibiting and/or degrading sirtuin (e.g., SIRT5) related desuccinylase activity;
inhibiting and/or degrading sirtuin (e.g., SIRT5) related demalonylase activity;
inhibiting and/or degrading sirtuin (e.g., SIRT5) related deglutarylase activity; and
influencing multiple cellular pathways related to sirtuin activity (e.g., SIRT5) such as ammonia detoxification, fatty acid oxidation, cellular respiration, ketone body formation, tricarboxylic acid cycle (TCA), glycolysis and reactive oxygen species (ROS) metabolism.

4. The compound of claim 1, wherein R¹ is selected from the group consisting of hydrogen, alkyl, cycloalkyl, heteroalkyl, heterocycloalkyl, aryl, heteroaryl, arylalkyl, and heteroarylalkyl.

5. The compound of claim 1, wherein R¹ is selected from the group consisting of hydrogen, methyl, ethyl, n-propyl, isopropyl, n-butyl, isobutyl, sec-butyl, tert-butyl, pentyl,

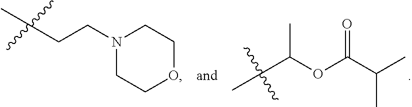

6. The compound of claim 1, wherein A is selected from the group consisting of NH, CH₂, O, S, 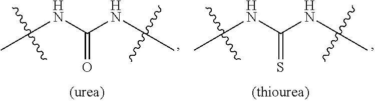 [SMILES: *NC(=O)N* ; *NC(=S)N*]
amide, and sulfonamide.

7. The compound of claim 1, wherein B is an aromatic ring.

8. The compound of claim 1, wherein B is a thiazole ring (e.g.

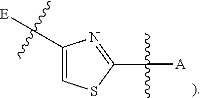

9. The compound of claim 1, wherein E is an aromatic ring.

10. The compound of claim 1, wherein E is a benzene ring.

11. The compound of claim 1, wherein 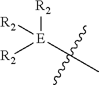
is selected from:

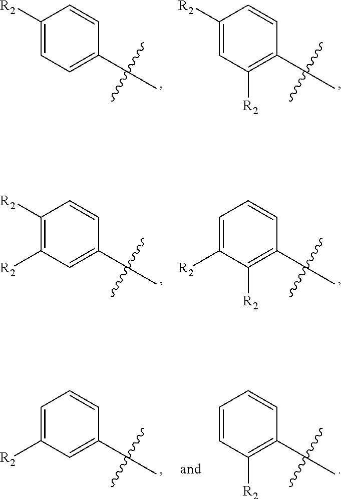

12. The compound of claim 1, wherein each R² within 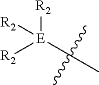
is independently selected from the group consisting of hydrogen, halogen (e.g., F, Cl, Br, I), alkyl, cycloalkyl, heteroalkyl, heterocycloalkyl, halogen, aryl, heteroaryl, arylalkyl, heteroarylalkyl, nitryl, cyano, amide or sulfonamide, 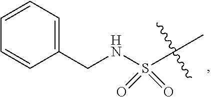 [SMILES: *S(=O)(=O)NCc1ccccc1]
and triphenylphosphine (TPP) attached with linker; and/or
wherein each R² within 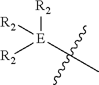
is independently selected from the group consisting of hydrogen, halogen, CF₃, OCH₃, OH, NO₂ (nitryl), CN (cyano), amide (e.g., NHC(O)CH₃), and sulfonamide (e.g., S(O2)NHCH3), 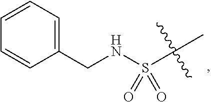 [SMILES: *S(=O)(=O)NCc1ccccc1]
and triphenylphosphine (TPP) group attached with a linker.

13. The compound of claim 12, wherein the linker is covalently bonded to connect two parts; wherein the carbon atom in the linear chain can be substituted with oxygen, nitrogen, sulfur, ester, and amide.

14. The compound of claim 12, wherein the linker is of following Formula (L0): 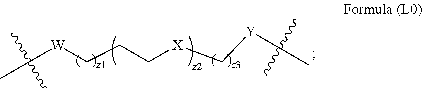
or an enantiomer, diastereomer, or stereoisomer thereof, wherein z1 is an integer selected from 0 to 10; Z2 is an integer selected from 0 to 10; Z3 is an integer selected from 0 to 10; each X is independently absent, CH₂, O, S, NH, NR₁₃; wherein W is selected from absent, O, NH, NR₁₃, —OCH₂C(O)NH—, —CH₂CH₂C(O)NH—, —CH₂C(O)NH— or —C(O)NH—; wherein Y is absent, O, NH, NR₁₃, —OCH₂C(O)NH—, —CH₂CH₂C(O)NH—, —CH₂C(O)NH— or —C(O)NH—; and each R13 is independently C₁-C₃ alkyl. The linker can be substituted with an alkyl, halide, phenyl, benzyl, aryl, alkylene or heterocycle group.

15. The compound of claim 12, wherein the linker is one of the following moieties:

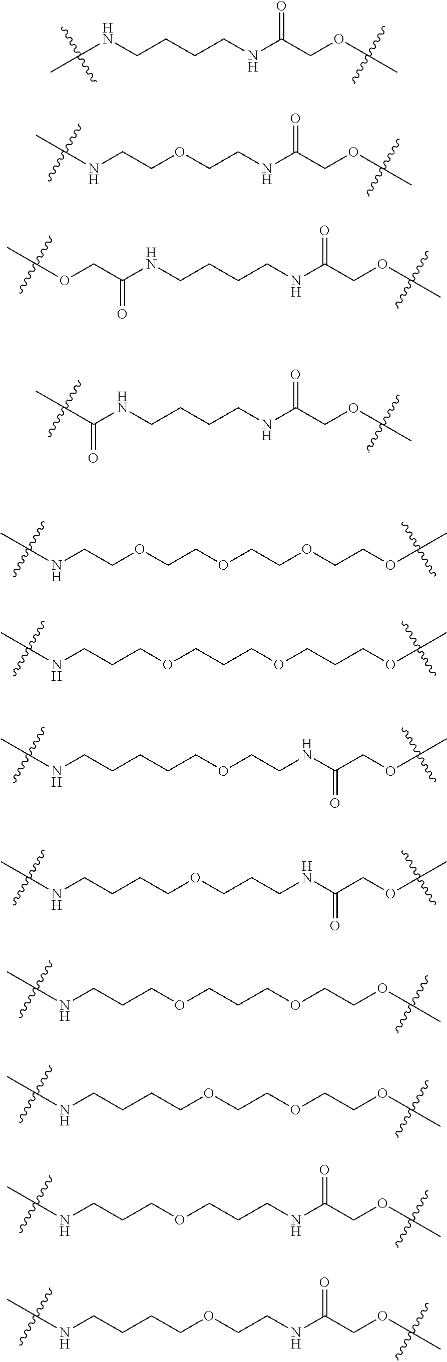

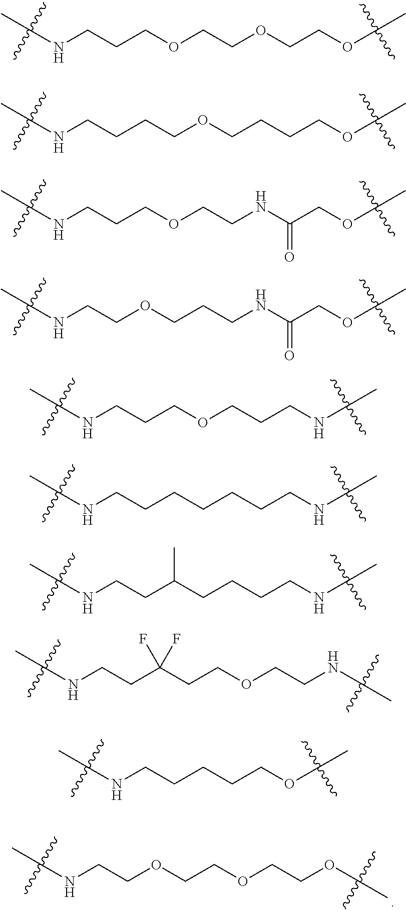

16. The compound of claim 1, wherein R² is represented by Formula (P0): 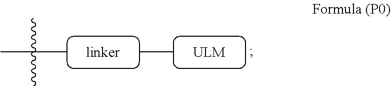
wherein the linker is covalently bonded to connect two parts; wherein the carbon atom in the linear chain can be substituted with oxygen, nitrogen, sulfur, ester, and amide; wherein ULM represents an E3 ubiquitin ligase binding moiety that binds E3 ubiquitin ligase selected from the group consisting of pomalidomide, thalidomide, lenalidomide, Von Hippel-Lindau (VHL), inhibitors of apoptosis proteins (IAP), Cereblon, and mouse double minute 2 (MDM2).

17. The compound of claim 1, wherein R3 is hydrogen or OH.

18. The compound of claim 1, wherein the compound is selected from one of the compounds recited in Table I.

19. A pharmaceutical composition comprising a compound recited in claim 1.

20. A method of treating, ameliorating, or preventing a disorder related to sirtuin (e.g., SIRT1, SIRT2, SIRT3, SIRT4, SIRT5, SIRT6, SIRT7) activity in a patient comprising administering to said patient a therapeutically effective amount of the pharmaceutical composition of claim 18.

21. The method of claim 20, wherein said disorder related to sirtuin (e.g., SIRT1, SIRT2, SIRT3, SIRT4, SIRT5, SIRT6, SIRT7) activity is cancer.

22. The method of claim 21, wherein the cancer is one or more of melanoma, Ewing sarcoma, malignant peripheral nerve sheath tumor, and non-small cell lung cancer.

23. The method of claim 20, wherein said patient is a human patient.

24. The method of claim 20, further comprising administering to said patient one or more agents for treating the disorder related to sirtuin (e.g., SIRT1, SIRT2, SIRT3, SIRT4, SIRT5, SIRT6, SIRT7) activity.

25. The method of claim 24, wherein the agents comprise anticancer agents, wherein said anticancer agent one or more of a chemotherapeutic agent, and radiation therapy.
